(19) United States
(12) Patent Application Publication
(10) Pub. No.: US 2019/0388559 A1
(43) Pub. Date: Dec. 26, 2019

(54) ENCAPSULATION OF METAL OXIDE NANOMATERIALS FOR CONTROLLED RELEASE AND TARGETED DELIVERY

(71) Applicant: Boise State University, Boise, ID (US)

(72) Inventors: Josh Eixenberger, Eagle, ID (US); Daniel Fologea, Boise, ID (US); Catherine Anders, Boise, ID (US); Denise Wingett, Boise, ID (US)

(21) Appl. No.: 16/451,967

(22) Filed: Jun. 25, 2019

Related U.S. Application Data
(60) Provisional application No. 62/689,528, filed on Jun. 25, 2018.

(51) Int. Cl. A61K 47/69; A61K 9/50; A61K 9/48; A61K 8/11; A61K 9/51; A61K 9/127; A61K 47/02

(52) U.S. Cl. CPC A61K 47/6925; A61K 9/5005; A61K 47/6911; A61K 9/4833; B82Y 5/00; A61K 9/4816; A61K 9/5107; A61K 9/1273; A61K 47/02; A61K 8/11

(57) ABSTRACT

The present invention is directed to micro and nanosized capsule compositions and methods of using and making the capsule compositions. The capsule compositions comprise an outer layer of lipids and/or polymers and inner contents comprising semiconductor nanoparticles. The nanoparticles are either metal oxides or quantum dots and will produce reactive oxygen species when irradiated with either electromagnetic radiation or ultrasound. The reactive oxygen species will degrade the outer layer of the capsule and cause the release of the contents, including the reactive oxygen species, into the local environment. The contents may optionally include cancer treating agent, water treating agents, antimicrobials, imaging and/or contracting agents. The outer layer may be further coated to protect it from environmental factors and/or be conjugated with a targeting molecule to increase delivery to a target.

### CROSS-REFERENCE TO RELATED APPLICATIONS

This application claims priority under 35 U.S.C. § 119 to provisional application U.S. Ser. No. 62/689,528, filed Jun. 25, 2018, herein incorporated by reference in its entirety.

### FIELD OF THE INVENTION

The present application relates to encapsulated nanoparticles coupled with agents capable of treating a variety of conditions, including methods relating thereto. More particularly, the present application relates to encapsulated nanoparticles which when exposed to irradiation will create reactive oxygen species which can lyse the capsule and release the agents into the local environment.

### BACKGROUND OF THE INVENTION

Agents used in treating cancer, water, or infection, or used for cosmetics can sometimes have severe off target toxicities. Off target toxicity is compounded by the use of these agents in bodies that are at times much larger than an agent's actual target. This large body may not only produce many off targets, but, due to the large size, also requires the use of a larger than needed amount of the agent just to reach the intended target.

Many semiconductor nanoparticles can generate reactive oxygen species when exposed to radiation, either light or sound, of the proper energy wavelength. The radiation promotes an electron from the valence band of the metal oxide to its conduction band, thereby creating a valence band hole. Conduction band electrons accumulate, which catalyze the reduction of oxygen to superoxide anion radicals and can subsequently lead to the production of hydrogen peroxide and then to hydroxyl radicals. Valence band holes migrate to the surface of the nanoparticle, where they remove electrons from water, hydroxide anions, or organic molecules to create hydroxyl radicals. There hydroxyl radicals can then form other reactive oxygen species.

However, one of the well-known physical properties and proposed toxicity mechanisms of some nanoparticles, such as ZnO nanoparticles, is particle dissolution. This feature is enhanced in lower pH environments, such as in certain tumors or intracellular compartments, and has been exploited as a means to control drug escape. An example of utilizing the dissolution potential of ZnO is to encapsulate doxorubicin loaded SiO₂ nanoparticles with a layer of ZnO. The ZnO layer prevents premature drug leakage, but when exposed to a lower pH, the quick dissolution of ZnO induces the release of the drug.

As the reactive oxygen species can cause oxidative stress, which can cause lipid peroxidation, using a lipid membrane around the nanoparticle may also prevent particle dissolution. When a lipid membrane undergoes peroxidation, the membrane can be damaged and may become perforated, allowing its contents to possibly diffuse into the local environment. It may be possible to induce metal oxide nanoparticles encapsulated within a lipid bilayer to produce enough reactive oxygen species to sufficiently damage the lipid bilayer capsule and cause enough disruption to release the reactive oxygen species, the metal oxide nanoparticles, and/or anything contained within the capsule into the local environment.

Additionally, some nanoparticles, such as ZnO, are prone to aggregation, especially in environments with high ionic concentrations such as in biological fluids. This may also impede their effectiveness as a therapeutic and thus many reports have looked at ways to achieve stable nZnO suspensions. A common strategy to stabilize nZnO in solution is to coat the particles with polymers or biomolecules. Lipids vesicles are already extensively used for drug delivery and may help increase the stability of nZnO while simultaneously preventing premature dissolution.

The use of lipid bilayers can further be used to enhance the targeting. Micelles and liposomes have been used to deliver therapeutic agents in cancers. This generally involves conjugation of the micelle or liposome with a targeting molecule which will then bind to the desired target. In cancer, these compositions are generally taken into the cell and will then take advantage of the pH imbalance found in tumors or the highly acidic lysosomes to cause the micelle or liposome to lyse and release its contents into the local environment. However, this system only works in instances where there is a pH imbalance within the local environment. If the pH is constant throughout the local environment, such as in contaminated water, another approach must be taken for the micelle or liposome to lyse. One such way may be to take advantage of the reactive oxygen species production of nanoparticles to cause sufficient damage to the surrounding lipid capsule to cause it to lyse and release its contents into the local environment. This would also allow the encapsulated nanoparticle to only have to associate with a target and not, for example, be required to be taken up into cells.

Accordingly, it is an aspect of the invention to develop compositions which can be used in a variety of large bodies to efficiently deliver agents to their intended targets. These compositions will allow for the reduction of both off target toxicities and the amount of agent required to achieve an effect.

Another aspect of the invention is to develop a pH independent composition that will still undergo a controlled and sufficient lysis to release its contents into the local environment.

A further aspect of the invention is to develop a composition that must merely associate with a target, such as a cell, and not have to be taken into said target to release its contents.

Another aspect of the invention is to develop methods to control the delivery of the composition's components into the local environment.

Other objects, advantages and features of the present invention will become apparent from the following specification taken in conjunction with the accompanying figures.

### BRIEF SUMMARY OF THE INVENTION

Applicants have created compositions of lipid and/or polymer encapsulated (“capsule”) nanoparticles and other, optional components, that have a triggered release of the components contained within the capsule through the generation of reactive oxygen species (“ROS”) by the nanoparticles upon either light or sound irradiation. The ROS will also be released with any other optional component within the capsule to confer their beneficial effects to a local environment. The nanoparticles may be either semiconductor or metallic oxide or any mixture thereof.

In an embodiment, a nanomaterial capable of producing reactive oxygen species when exposed to radiation is encapsulated within a lipid bilayer or polymer capsule. The capsule can be made from any lipid bilayer, including, but not limited to, naturally or synthetically derived lipids, a cellular membrane, and/or in a polymer matrix.

In a further embodiment, the capsule contains both a nanomaterial and an additional agent, such as but not limited to an antibiotic, a cytotoxic or cytostatic agent, salts or acids for water treatment, photosensitizers, imaging contrast agents and/or cosmetics. The additional agent would get released from the capsule after the metal oxide nanoparticle was irradiated, causing the capsule to lyse from the creation of ROS.

Another further embodiment, the nanomaterials are associated with other particles, such as but not limited to gold, silver, phosphate, or boron, or a microbubble. This may increase the efficiency of ROS production by allowing the absorbing of wavelengths at different or multiple frequencies.

In another embodiment, the capsule is conjugated with a targeting molecule which enhances the capsules association with a target. The targeting molecule could include, but not limited to, antibodies or receptor ligands.

In yet a further embodiment, the capsule comprises a coating around the capsule to help protect it in some body. This coating could include, but not limited to, polyethylene glycol or additional lipid membranes.

In another embodiment, the compositions are administered to a large body containing a target, dissipate through the body, and associate with the compositions' target. The body or target is then exposed to the appropriate radiation, the metal oxide nanomaterial produces ROS, the ROS lyse the lipid bilayer capsule, releasing the contents contained within the capsule into the local environment.

In another embodiment, lipid bilayer and nanoparticles are mixed together in a method to encapsulate the nanoparticles within the lipid bilayer. In a further method of encapsulation, agents are additionally encapsulated within the lipid bilayer. In yet a further method of encapsulation, an agent is additionally encapsulated within the lipid bilayer and additional agent(s) are encapsulated within the capsule.

While multiple embodiments are disclosed, still other embodiments of the present invention will become apparent to those skilled in the art from the following detailed descriptions, which show and describe illustrative embodiments of the invention. Accordingly, the figures and detailed description are to be regarded as illustrative in nature and not restrictive.

### BRIEF DESCRIPTION OF THE DRAWINGS

FIG. 1 is a schematic representation of the photo irradiation of an exemplar metallic oxide nanoparticle comprised of zinc oxide. The metallic oxide nanoparticle is encapsulated with a lipid bilayer or other matrix. Upon photo irradiation, the metallic oxide nanoparticle creates reactive oxygen species (ROS) which disrupt the capsule, releasing the contents into the local environment. Additional agents may be encapsulated along with the metallic oxide nanoparticles or within the lipid bilayer or the capsule depending on their hydrophilicity or hydrophobicity.

FIG. 2A is a graphical representation of the XRD pattern of the zinc oxide nanoparticle (nZnO) synthesis as in Example 1 having a calculated average crystal size of 15 nm. FIG. 2B is a photographic representation of a TEM image of the aggregates composed of the smaller nZnO crystals (scale bar is 50 nm). FIG. 2C is a photographic representation of the electron diffraction pattern of the nZnO (scale bar is 5/2 nm).

FIG. 3A is a graphical representation of the bare nZnO size distribution from DLS measurements suspended in nanopure water having a broad distribution between about 100 and 1000 nm. FIG. 3B is a graphical representation of the bare nZnO size distribution suspended in 130 mM NaCl showing agglomeration into much larger micrometer range.

FIG. 3C is a graphical representation of the encapsulated nZnO size distribution in 130 mM NaCl showing the capsule prevents the NPs from agglomerating into the large particles even in the 130 mM naCl solution, having a size distribution more similar to bare nZnO in nanopure water.

FIG. 4A is a graphical representation of the XPS scan of the NPs before encapsulation showing no other elements beside zinc, oxygen, and carbon are present. FIG. 4B is a graphical representation of the FTIR spectra confirming the sample purity seen with XPS measurements with the main peak near 500 cm⁻¹ from the Zn—O modes, the broad peak at 3400 cm⁻¹ being due to O—H bonds, with the other minor peaks arising from carbon dioxide.

FIG. 5 is a graphical representation of the self-quenching curve of 5(6)-carboxyfluorescein in 130 mM NaCl showing a linear increase in intensity vs. concentration below 1.5 μM.

FIG. 6A is a photographical representation of a confocal microscopy image of fluorescent nZnO excited by a 405 nm laser. FIG. 6B is a photographical representation of lipid membranes stained with CellMask Orange. FIG. 6C is the overlay image of 6A and 6B, demonstrating efficient encapsulation of the nZnO.

FIG. 7A is a graphical representation of Jurkat T cell leukemia cell viability with treatment of different concentrations of free, non-encapsulated, nZnO with or without a UV treatment. FIG. 7B is a graphical representation of Jurkat T cell leukemia cell viability with treatment of with difference concentrations of encapsulated nZnO with or without UV treatment. FIG. 7C is a graphical representation of T47D breast cancer cell viability with treatment of different concentrations of encapsulated nZnO with or without UV treatment.

FIG. 7B and FIG. 7C also demonstrate that the non-UV groups (left bars) show that the membrane encapsulation of nZnO protects the cells from the toxic effects of the NPs. The UV treatment groups (right bars) demonstrates that the toxic effects of nZnO are reestablished by the triggered release of the particles, due to ROS generated from the nZnO and subsequent shedding of the lipid coating. UV exposure=3 minutes for Jurkat T cells; 2 minutes for T47D cells.

FIG. 8 is a photographical representation of confocal images of T47D breast cancer cells after 48 hours of incubation in the imaging chambers and 24 hours post nZnO treatment. (Top Row) T47D cells incubated in the absence of nZnO (negative control) to show lack of auto-fluorescence from the cells in the nZnO channel. The lipid membrane stain, CellMask Orange was utilized to demonstrate the normal morphology of the cells (top-middle panel). (Middle Row) T47D cells treated with 81.4 μg/mL (1 mM) of the encapsulated nZnO (Enc-nZnO) 24 hours prior to imaging. These images demonstrate that even at relatively high concentrations of the Enc-nZnO, the cells morphology doesn't appear to be affected suggesting that the toxicity of the NPs is negligible. (Bottom Row) T47D cells treated with 20.3 μg/mL (250 μM) of Enc-nZnO and irradiated for 2 minutes shows changes in the confluency of the cells and an apparent loss of cell membrane integrity.

FIG. 9 is a graphical representation showing the controlled release of the encapsulated agent and nanoparticles due to photo irradiation and the minimal release of the encapsulated agent without photo irradiation.

FIG. 10A is a graphical representation of the release kinetics of encapsulated 5(6)-carboxyfluoroscein (pH=7.4) and nZnO with various lipid to nZnO ratios (w/w) without UV treatment, showing premature drug leakage from the different lipid to nZnO ratios. FIG. 10B is a graphical representation of the release kinetics after 5 minutes of UV exposure. FIG. 10C is a graphical representation of the release kinetics after 15 minutes of UV exposure. These release profiles demonstrate the majority of the contents can be rapidly released within 60 minutes and provides insights into the optimal lipid to NPs ratio. FIG. 10D is a graphical representation of the release kinetics of nZnO encapsulated with a 5:4 lipid to NP ratio with an internal pH=9.85 at various UV exposure times. The higher pH is believed to allow the nZnO to produce more ROS, thus allowing for a faster release of the dye with less irradiation time.

FIG. 11A is a graphical representation of the viability of Jurkat T cells after 48 hours of treatment with Paclitaxel (PTX) co-loaded with the Enc-nZnO (Enc-nZnO/PTX) compared to the viability profile of the Jurkat cells when treated with free Paclitaxel and relatively higher nZnO to PTX ratios. Comparing the released (UV Tx) vs. the non-released (No UV) an increase in toxicity is noted for every concentration tested. FIG. 11B is a graphical representation of the viability of Jurkat T cells after 48 hours of treatment with Paclitaxel (PTX) co-loaded with the Enc-nZnO (Enc-nZnO/PTX) with relatively lower nZnO to PTX ratios demonstrating that increased toxicity is still observed in the triggered release group at concentrations of nZnO that do not impact cell viability when used alone. FIG. 11C is a graphical representation of the viability of Jurkat T cells after 48 hours of treatment with Paclitaxel (PTX) co-loaded with the Enc-nZnO (Enc-nZnO/PTX) showing treatments with the free drug PTX (5.0 nM), free nZnO (8.1 μg/mL) and free nZnO/PTX (8.1 μg/mL-5.0 nM) compared with the Enc-nZnO/PTX (High nZnO 8.1 μg/mL; Low nZnO 2.0 μg/mL; PTX 5.0 nM) with and without UV exposure.

FIG. 12A is a graphical representation of the viability profile of T47D breast cancer cells after 48 hours of treatment of Enc-nZnO/PTX with and without UV irradiation demonstrating the viability profile of the triggered release group vs. the non-irradiated group showing a striking difference in toxicity. FIG. 12B is a graphical representation of the viability profile of T47D breast cancer cells after 48 hours of treatment of Enc-nZnO/PTX with and without UV irradiation demonstrating treatments with free nZnO (16.3 μg/mL), free PTX (40 nM) and free nZnO/PTX (16.3 μg/mL-40 nM) compared to the Enc-nZnO/PTX (16.3 μg/mL-40 nM) that at the 16.3 μg/mL concentration of nZnO, the NPs/PTX have no added or synergistic effects when the T47D cells are challenged with both the particles and PTX and a drastic improvement in the drug's efficacy is noted in the triggered release group, due to localized release of the drug in the vicinity of the cells.

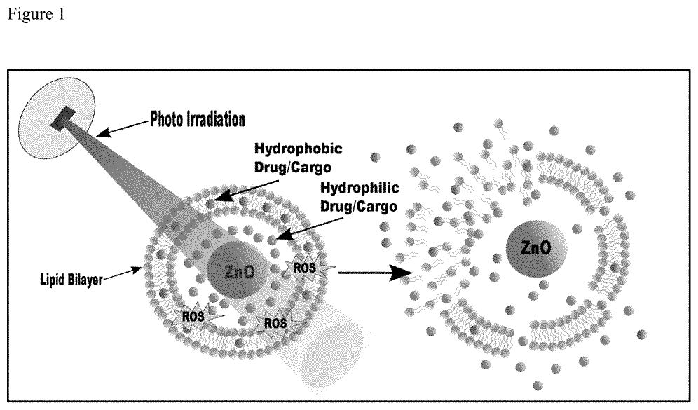

### DETAILED DESCRIPTION

Unless otherwise defined herein, scientific and technical terms used in connection with the invention shall have the meanings that are commonly understood by those of ordinary skill in the art. Further, unless otherwise required by context, singular terms shall include the plural and plural terms shall include the singular. Generally, nomenclatures used in connection with, and techniques of, biochemistry, enzymology, molecular and cellular biology, microbiology, genetics and protein and nucleic acid chemistry and hybridization described herein are those well-known and commonly used in the art. The methods and techniques are generally performed according to conventional methods well known in the art and as described in various general and more specific references that are cited and discussed throughout the present specification unless otherwise indicated. See, e.g., Sambrook et al. Molecular Cloning: A Laboratory Manual, 2d ed., Cold Spring Harbor Laboratory Press, Cold Spring Harbor, N.Y. (1989); Ausubel et al., Current Protocols in Molecular Biology, Greene Publishing Associates (1992, and Supplements to 2002); Harlow and Lane, Antibodies: A Laboratory Manual, Cold Spring Harbor Laboratory Press, Cold Spring Harbor, N.Y. (1990); Taylor and Drickamer, Introduction to Glycobiology, Oxford Univ. Press (2003); Worthington Enzyme Manual, Worthington Biochemical Corp., Freehold, N.J.; Handbook of Biochemistry: Section A Proteins, Vol. I, CRC Press (1976); Handbook of Biochemistry: Section A Proteins, Vol. II, CRC Press (1976); Essentials of Glycobiology, Cold Spring Harbor Laboratory Press (1999), which are incorporated herein by reference.

The following terms, unless otherwise indicated, shall be understood to have the following meanings:

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

Numeric ranges recited within the specification are inclusive of the numbers defining the range and include each integer within the defined range. Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾. This applies regardless of the breadth of the range.

Other than in the operating examples, or where otherwise indicated, all numbers expressing quantities of ingredients or reaction conditions used herein are to be understood as being modified in all instances by the term “about”.

As used herein, the term “about” modifying the quantity of an ingredient in the compositions of the invention or employed in the methods of the invention refers to variation in the numerical quantity that can occur, for example, through typical measuring and liquid handling procedures used for making concentrates or use solutions in the real world; through inadvertent error in these procedures; through differences in the manufacture, source, or purity of the ingredients employed to make the compositions or carry out the methods; and the like. The term about also encompasses amounts that differ due to different equilibrium conditions for a composition resulting from a particular initial mixture. Whether or not modified by the term “about,” the claims include equivalents to the quantities.

“Non-covalent” refers to any molecular interactions that are not covalent—i.e. the interaction does not involve the sharing of electrons. The term includes, for example, electrostatic, 2-effects, van der Waals forces, and hydrophobic effects. “Covalent” refers to interactions involving the sharing of one or more electrons.

As used herein, the term “agent” refers to a substance used in the diagnosis, treatment, or prevention of a disease; antibiotics; a substance used for the treatment of water; photosensitizers; or a substance used in cosmetics.

As used herein, a “metal oxide” is any metal oxide that can produce reactive oxygen species when exposed to certain wavelengths of radiation. The metal oxide includes, but is not limited to, MgO, CaO, TiO₂, CeO₂, ZrO₂, FeO, V₂O₅, Mn₂O₃, Fe₂O₃, NiO, CuO, Al₂O₃, SrO, BaO, V₂O₃, Ag₂O, HfO₂, and ZnO. As used herein, “metallic oxide” may be used interchangeably with “metal oxide.”

As used herein, “semiconductor” is a solid substance with a conductivity between an insulator and that of most metal, typically due to the inclusion of impurities, such as oxygen, in the metal. By way of nonlimiting example metal oxides and quantum dots are both semiconductors.

As used herein, “metallic nanoparticles” are nanosized particles having a metal core or nanoparticles containing a metal. “Metallic oxide nanoparticles” are nanosized particles having a metal oxide core or nanoparticle containing a metal oxide.

As used herein, “nanomaterial” is any material having one or more constituents of nanosized dimensions. A “metallic oxide nanomaterial” are nanosized particles containing a metal oxide and one or more constituents, for example a nanoparticle comprising of a mesoporous silica core coated with a metal oxide.

As used herein, an “association particle” is a metal particle which may have a plasmon effect with the metal oxide of a metallic nanoparticle.

As used herein, a “capsule” is any lipid or matrix which surrounds a metallic nanoparticle and/or agent, encapsulating at least the metallic nanoparticle, and is suitable to be disrupted by reactive oxygen species. The capsule may further be conjugated to a targeting molecule, targeting moiety, or targeting ligand to increase its targeting. A capsule may also contain other constituents, such as metallic nanoparticles and/or agents.

As used herein, a “coating” surrounds the capsule and may aid increase the half-life of the capsule in a body by protecting the capsule from environmental factors.

As used herein, a “photosensitizer” is a molecule that may enhance the reactive oxygen species production of the metallic oxide.

As used herein, a “target” is the object in which the nanoparticles and/or agents may affect, and such affect results in a desirable outcome as described herein.

As used herein, an “off target” is an object that the nanoparticles and/or agents may affect, but such an effect would have detrimental results.

As used herein the term “targeting molecule,” “targeting moiety,” or “targeting ligand” refers to any molecule that provides an enhanced affinity for a selected target, e.g., a cell, cell type, tissue, organ, region of the body, a compartment, or other compound, e.g., a cellular, tissue, organ compartment, bacteria, or water contaminate. The targeting moiety or ligand can comprise a wide variety of entities. Such ligands can include naturally occurring molecules, or recombinant or synthetic molecules.

More specifically, a “targeting molecule,” “targeting moiety,” or “targeting ligand” may be a protein or non-protein molecule which is characterized by selective localization to an organ, tissue, cell type, peptide, or antigen.

As used herein the term “linker” means an organic moiety that connects two parts of a compound. Linkers typically comprise a direct bond or an atom such as oxygen or sulfur, a unit such as NH, C(O), C(O)NH, SO, SO₂, SO₂NH, or a chain of atoms, such as substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, where one or more methylenes can be interrupted or terminated by O, S, S(O), SO₂, NH, C(O). The terms linker and spacer are used interchangeably herein. The linker can comprise any combinations of the above.

As used herein, a “body” is any place where fluid, powder, or gel collects or flows. Such places may be, but not limited to, body cavities, interstitial space, or vessels; rivers, lakes, or smaller or larger bodies of water; or a container for powders, lotions, or gels.

As used herein, a “local environment” is the area surrounding the encapsulated nanoparticle in which the released components may have an effect.

As used herein, the term “administer” or “administering” refers to the placement of a composition into a body by a method or route which results in at least partial localization of the composition at a desired local environment or target such that desired effect is produced. A compound or composition described herein can be administered in a human or animal body by any appropriate route known in the art including, but not limited to, oral or parenteral routes, including intravenous, intramuscular, subcutaneous, transdermal, airway (aerosol), pulmonary, nasal, rectal, and topical (including buccal and sublingual) administration. Further routes of administration can include, but not limited to, upstream of a desired target in a water body or in such a way that the composition may reach the desired target.

As used herein, the term “substantially free” refers to compositions completely lacking the component or having such a small amount of the component that the component does not affect the effectiveness of the composition. The component may be present as an impurity or as a contaminant.

As used here, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

The phrase “therapeutically-effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment. For example, an amount of a compound administered to a subject that is sufficient to produce a statistically significant, measurable change in at least one symptom of cancer or metastasis.

By “treatment”, “prevention” or “amelioration” of an adverse condition is meant delaying or preventing the onset of such a disease or disorder, reversing, alleviating, ameliorating, inhibiting, slowing down or stopping the progression, aggravation or deterioration the progression or severity of a condition associated with such an adverse condition. In one embodiment, at least one symptom of an adverse condition is alleviated by at least 5%, at least 10%, at least 20%, at least 30%, at least 40%, or at least 50%.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Other animals may include ferrets or rabbits. Patient or subject includes any subset of the foregoing, e.g., all of the above, but excluding one or more groups or species such as humans, primates or rodents. In certain embodiments, the subject is a mammal, e.g., a primate, e.g., a human. The terms, “patient” and “subject” are used interchangeably herein.

As used herein, “w/w” is the ratio of the weight of one compound or composition compared the weight of another compound or composition.

### Reactive Oxygen Species Generating Nanoparticles

The nanoparticles or nanomaterials are made of or contain at least one semiconductor material, such as a metallic oxide, where the semiconductor may produce reactive oxygen species (ROS) when exposed to radiation or ultrasound may be used. Semiconductors include quantum dots (QD) and metal oxides. Preferred quantum dots include, but not limited to, core-type quantum dots, core-shell quantum dots, or alloyed quantum dots and may be comprised of Si, Ge, CdTe, PbS, PbS₂, CdSe, CdS, InAs, InP, PbSe, CuInS, ZnS, CdSₓSe₁₋ₓ, or graphene. In some embodiments, mixtures of quantum dot types or materials may be used. Preferred metal oxides include, but not limited to, MgO, CaO, TiO₂, CeO₂, ZrO₂, FeO, V₂O₅, Mn₂O₃, Fe₂O₃, NiO, CuO, Al₂O₃, SrO, BaO, V₂O₃, Ag₂O, HfO₂, and ZnO. While not a metallic oxide, SiO₂ may also be used. In some embodiments, mixtures of the metal oxides may be used. In some embodiments, nanomaterial mixtures of semiconductors including quantum dots and metal oxides may be used. In further embodiments, the mixtures may be mixtures of semiconductor types or materials and a single metal oxide or a single semiconductor and a mixture of metal oxides, or a mixture of semiconductor types or materials and mixtures of metal oxides.

The nanomaterials may be made with any of the various methods, or variations thereof, of making nanomaterials known in the art. Any of the various methods may be used to synthesis the quantum dots including, but not limited to colloidal synthesis, plasma synthesis, viral assembly, electrochemical assembly, high temperature dual injection synthesis, self-assembly fabrication, spontaneous formation in a quantum well fabrication, two-dimensional electron or hole gas fabrication, or complementary metal-oxide-semiconductor (CMOS) fabrication. Any of the various methods may be used to synthesis the metallic oxide nanomaterials, including, but not limited to forced hydrolysis, aerogel techniques, chemical solution techniques, laser pyrolysis techniques, autoclave treatment methods, inert or dynamic gas condensation methods, mixing and reflux of metallic salts, plasma-based processes, biosynthesis methods, electrochemical methods, gas-solid transformation methods, solution-phase methods, galvanic replacement, and/or polyol process.

The nanomaterial will preferably produce ROS when exposed to its corresponding wavelength of radiation or frequency and power of ultrasound. The wavelength of the radiation will depend on the material used in the quantum dot or the metal used in the metallic oxide and would be in the electromagnetic range of about 1 fm to 100 μm. More specifically, in the ranges of about 1 fm to about 10 μm (gamma rays), about 6 μm to about 200 μm (hard x-rays), about 200 μm to about 10 nm (soft x-rays), about 6 nm to about 400 nm (ultraviolet light), about 380 nm to about 760 nm (visible light), about 750 nm to about 2.5 μm (near infrared), about 2.5 μm to about 10 μm (medium infrared), or about 10 μm to about 100 μm (far infrared). Some metallic oxide can be exposed to multiple ranges of wavelengths to produce ROS, such as across about 6 nm to about 760 nm, the ultraviolet to visible light spectrum, or across about 6 μm to about 400 nm, the x-ray to ultraviolet spectrum.

The nanoparticles may be exposed to ultrasound radiation at either high or low intensity. High intensity ultrasound may be preferably applied in any frequency and power output which may generate ROS, for example frequencies ranging from about 0.1 to about 3 MHz, from about 0.2 to about 2.5 MHz, from about 0.3 to about 2 MHz with a power output between about 5 W to about 2,000 W, from about 10 W to about 1,800 W, or from about 15 to 1,700 W. The ultrasound is preferably applied to a limited area when the body is a human or animal to reduce damaging surrounding tissue and in either a single exposure or in cycles. Low intensity ultrasound may be applied in any frequencies and power output which may generate ROS, for example from about 0.2 MHz to about 6 MHz, from about 0.3 MHz to about 5 MHz, from about 0.5 MHz to about 3.5 MHz, or from about 1 MHz to about 2 MHz at a power output from about 0.02 Wcm⁻² to about 600 Wcm⁻², from about 0.2 Wcm⁻² to about 300 Wcm⁻², or from about 0.5 Wcm⁻² to about 10 Wcm⁻². The ultrasound may be applied all at once or in cycles.

The nanoparticles may be irradiated with either a targeted stream or the entire body may be irradiated. The targeted stream may be directed to the vicinity of an intended target. Any method can be used to deliver the appropriate wavelength or frequency and power output of radiation, such as, but not limited to, polychromatic polarized light, lasers, light-emitting diodes, fluorescent lamps, dichroic lamps, linear accelerators, radiation source projectors, bright, full-spectrum light, and/or an ultrasound generating apparatus.

In other embodiments at least one association particle may be doped into the nanoparticle. The association particle is preferably gold, silver, iron, gadolinium, and/or any lanthanide and may increase the range of the radiation's wavelength which the metallic oxide may produce ROS. In other embodiments, quantum dots may be doped to control their optical and electrical properties. Preferred dopants include, but are not limited to, boron and phosphate. Any method known in the art may be used to dope the nanoparticle.

### Lipid or Matrix Capsule

Capsule material may consist of any lipid bilayer either from naturally or synthetically derived lipids, a cellular membrane, or a polymer matrix. The capsule can be made of any lipid, such as, but not limited to, one or more cationic, neutral, or anionic lipid, a synthetic, semi-synthetic, or naturally occurring lipids, including phospholipids, tocopherols, sterols, fatty acids, and/or glycoproteins, such as albumin. Representative examples of lipid components of the capsule include, but are not limited to, one or more of phosphatidylcholine, cholesterol, alpha-to-copherol, dipalmitoylposphatidylcholine, and/or phosphatidyl serine. The capsule may also comprise of naturally occurring cellular membranes from prokaryotes or eukaryotes. The different components will provide different properties to the capsule, such as binding to targeting molecules, encapsulating various agents, preventing immune system clearance, or aiding in the targeting and uptake into certain cells.

Usable phospholipids include, but are not limited to, egg phosphatidylcholine, egg phosphatidylglyercol, egg phosphatidylinositol, egg phosphatidylserine, phosphatidylethanolamine, phosphatidic acid, soya counter parts, soy phosphatidylcholine, the hydrogenated egg and soya counterparts, other phospholipids made up of ester linkages of fatty acids in the 2 and 3 of glycerol position containing chains of 12 to 26 carbon atoms and different head groups in the number 1 position of glycerol that include choline, glycerol, inositol, serine, ethanolamine, and the corresponding phosphatidic acids. The chains can be saturated or unsaturated, and the phospholipid may be made up of fatty acids of different chain lengths and different degrees of unsaturation.

The sterols include, but are not limited to, cholesterol, esters of cholesterol including cholesterol hemi-succinate, salts of cholesterol including cholesterol hydrogen sulfate and cholesterol sulfate, ergosterol, esters of ergosterol including ergosterol hemi-succinate, salts of ergosterol including ergosterol hydrogen sulfate and ergosterol sulfate, lanosterol, esters of lanosterol including lanosterol hemi-succinate, salts of lanosterol including lanosterol hydrogen sulfate and lanosterol sulfate. The tocopherols can include tocopherols, esters of tocopherols including tocopherol hemi-succinates, salts of tocopherols including tocopherol hydrogen sulfates and tocopherol sulfates. One or more sterols could be used in the capsule.

The cationic lipids used can include, but are not limited to, ammonium slats of fatty acids, phospholipids, and glycerides. The fatty acids include fatty acids of carbon chain lengths preferably of 12 to 26 carbon atoms that are either saturated or unsaturated. The anionic lipids used can include, but are not limited to, phosphatidyl-glycerols, phosphatidic acids, phosphatidylinositols, and/or phosphatidyl serines.

The capsule may also be made of any polymer such as, but not limited to, polymers of carbohydrates, fatty acids, peptides, proteins, and/or nucleic acids monomers. The capsule can also be made from one or more lipids or one or more monomer.

The capsule may also be made of any polymer comprised of or including linkers sensitive to ROS such as, but not limited to, thioether, selenide, diselenide, telluride, thioketal, arylboronic ester, aminoacrylate, oligoproline, peroxalate ester, and/or mesoporous silicon.

Preparation of the capsule can be prepared by various methods, including, but not limited to, lyophilization of a frozen lipid film dissolved in volatile solvent, slow hydration of a thin layer of lipid by an aqueous solution, addition of an aqueous solution to a lipid film while shaking, emulsion stabilization, electrospray, in situ polymerization, extrusion methods, reverse phase evaporation, infusion procedures, detergent dilution, and/or sonication.

Various ratios of lipid and other components in the capsule may be used in the different embodiments. The ratio of lipids to other components will depend on the desired size of the nanoparticles and purpose of the encapsulated nanomaterial and/or agent. As the size of nanoparticle increases, the surface area made up of lipids may greatly increase in comparison to the other components.

The capsule may be disrupted when exposed to radiation in a sufficient amount to release any nanoparticles and/or agents contained within. This allows for the controlled release of high concentrations of ROS and/or agents into a local environment to affect a target.

To enhance targeting in some embodiments, the capsule may further be conjugated or covalently bound to at least one targeting molecule. The targeting molecule may either be integrated directly into the capsule or the lipids making up the capsule may be bound to a linker which is further bound to targeting molecule.

Exemplary targeting molecules include, but are not limited to, antibodies, antigen binding fragments of antibodies, antigens, folates, EGF, albumin, receptor ligands, carbohydrates, aptamers, integrin receptor ligands, chemokine receptor ligands, transferrin, biotin, serotonin receptor ligands, PSMA, endothelin, GCPII, somatostatin, LDL and HDL ligands. Additional exemplary targeting molecules include, but are not limited to, polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (e.g., PEG-2K, PEG-5K, PEG-10K, PEG-12K, PEG-15K, PEG-20K, PEG-40K), MPEG, [MPEG]2, polyvinyl alcohol (PVA), polyurethane, poly(2-ethylacryllic acid), N-isopropylacrylamide polymers, polyphosphazine, polyethylenimine, cationic groups, spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, thyrotropin, melanotropin, lectin, glycoprotein, surfactant protein A, mucin, glycosylated polyaminoacids, transferrin, bisphosphonate, polyglutamate, polyaspartate, aptamer, asialofetuin, hyaluronan, procollagen, immunoglobulins (e.g., antibodies), insulin, transferrin, albumin, sugar-albumin conjugates, intercalating agents (e.g., acridines), cross-linkers (e.g. psoralen, mitomycin C), porphyrins (e.g., TPPC4, texaphyrin, Sapphyrin), polycyclic aromatic hydrocarbons (e.g., phenazine, dihydrophenazine), artificial endonucleases (e.g., EDTA), lipophilic molecules (e.g, steroids, bile acids, cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine), peptides (e.g., an alpha helical peptide, amphipathic peptide, RGD peptide, cell permeation peptide, endosomolytic/fusogenic peptide), alkylating agents, phosphate, amino, mercapto, polyamino, alkyl, substituted alkyl, radiolabeled markers, enzymes, haptens (e.g. biotin), transport/absorption facilitators (e.g., naproxen, aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, AP, antibodies, hormones and hormone receptors, lectins, carbohydrates, multivalent carbohydrates, vitamins (e.g., vitamin A, vitamin E, vitamin K, vitamin B, e.g., folic acid, B12, riboflavin, biotin and pyridoxal), vitamin cofactors, lipopolysaccharide, an activator of p38 MAP kinase, an activator of NF-κB, taxon, vincristine, vinblastine, cytochalasin, nocodazole, japlakinolide, latrunculin A, phalloidin, swinholide A, indanocine, myoservin, tumor necrosis factor alpha (TNF-alpha), interleukin-1 beta, gamma interferon, natural or recombinant low density lipoprotein (LDL), natural or recombinant high-density lipoprotein (HDL), and a cell-permeation agent (e.g., α-helical cell-permeation agent).

Peptide and peptidomimetic targeting molecules include those having naturally occurring or modified peptides, e.g., D or L peptides; α, β, or γ peptides; N-methyl peptides; azapeptides; peptides having one or more amide, i.e., peptide, linkages replaced with one or more urea, thiourea, carbamate, or sulfonyl urea linkages; or cyclic peptides. A peptidomimetic (also referred to herein as an oligopeptidomimetic) is a molecule capable of folding into a defined three-dimensional structure similar to a natural peptide. The peptide or peptidomimetic ligand can be about 2-50 amino acids long, e.g., about 5, 10, 15, 20, 25, 30, 35, 40, 45, or 50 amino acids long.

Carbohydrate based targeting molecules include, but are not limited to, D-galactose, multivalent galactose, N-acetyl-D-galactose (GalNAc), multivalent GalNAc, e.g. GalNAc2 and GalNAc3; D-mannose, multivalent mannose, multivalent lactose, N-acetyl-galactosamine, N-acetyl-gulucosamine, multivalent fucose, glycosylated polyaminoacids and lectins. The term multivalent indicates that more than one monosaccharide unit is present. Such monosaccharide subunits can be linked to each other through glycosidic linkages or linked to a scaffold molecule.

A number of folate and folate analogs amenable to the present invention as targeting molecules are described in U.S. Pat. Nos. 2,816,110; 5,552,545; 6,335,434 and 7,128,893, contents of all of which are herein incorporated in their entireties by reference.

In some embodiments, the targeting molecule binds a protein, receptor, or marker expressed on the surface of a cancer cell.

In some embodiments, the targeting molecule is a polyclonal or monoclonal antibody or a fragment thereof retaining epitope binding activity or an antibody-based binding moiety.

In some embodiments, the targeting molecule is a polyclonal or monoclonal antibody, antibody fragments, a peptide, or a molecule that is capable of binding to the surface of cells. The binding could be to protein receptors on the surface of the cells. The cells may be cancer, bacterial, or fungal.

In some embodiments, the linker can comprise hydrocarbons, amino acids, peptides, polyethylene glycol of various lengths, cyclodextrins, and derivatives and any combinations thereof.

In some embodiments, the linker is a branched linker. By a branched linker is meant a linker that can connect together three or more parts together. The branch-point of the branched linker may be at least trivalent, but can be a tetravalent, pentavalent or hexavalent atom, or a group presenting such multiple valences. In some embodiments, the branchpoint is —N, —N(Q)-C, —O—C, —S—C, —SS—C, —C(O)N(Q)-C, —OC(O)N(Q)-C, —N(Q)C(O)—C, or —N(Q)C(O)O—C; wherein Q is independently for each occurrence H or optionally substituted alkyl. In some embodiments, the branch-point is glycerol or derivative thereof, and normal chain sugars such as monosaccharides and polysaccharides. A branched linker can be used to connect two or more (e.g., 2, 3, 4, 5, 6, 7, 8, 9, 10 or more) molecules of interest (which can be same or different) to the capsule; two or more (e.g., 2, 3, 4, 5, 6, 7, 8, 9, 10 or more) targeting molecules (which can be same or different) to one molecule of interest; or two or more (e.g., 2, 3, 4, 5, 6, 7, 8, 9, 10 or more) molecules of interest (which can be same or different) to two or more (e.g., 2, 3, 4, 5, 6, 7, 8, 9, 10 or more) targeting molecules (which can be same or different).

In some embodiments, the linker comprises at least one cleavable linking group. A cleavable linking group is one which is sufficiently stable outside the cell, but which upon entry into a target cell is cleaved to release the two parts the linker is holding together. In a preferred embodiment, the cleavable linking group is cleaved at least 10 times or more, preferably at least 100 times faster in the target cell or under a first reference condition (which can, e.g., be selected to mimic or represent intracellular conditions) than in the blood or serum of a subject, or under a second reference condition (which can, e.g., be selected to mimic or represent conditions found in the blood or serum).

Cleavable linking groups are susceptible to cleavage agents, e.g., pH, redox potential or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable linking group by reduction; esterases; amidases; endosomes or agents that can create an acidic environment, e.g., those that result in a pH of five or lower; enzymes that can hydrolyze or degrade an acid cleavable linking group by acting as a general acid, peptidases (which can be substrate specific) and proteases, and phosphatases. The cleavable linking group can comprise esters, peptides, carbamates, acid-labile, reduction-labile, oxidation-labile, disulfides, and modifications thereof.

A linker can include a cleavable linking group that is cleavable by a particular enzyme. The type of cleavable linking group incorporated into a linker can depend on the cell to be targeted. In some embodiments, cleavable linking group is cleaved at least 1.25, 1.5, 1.75, 2, 3, 4, 5, 10, 25, 50, or 100 times faster in the cell (or under in vitro conditions selected to mimic intracellular conditions) as compared to blood or serum (or under in vitro conditions selected to mimic extracellular conditions). In some embodiments, the cleavable linking group is cleaved by less than 90%, 80%, 70%, 60%, 50%, 40%, 30%, 20%, 10%, 5%, or 1% in the blood (or in vitro conditions selected to mimic extracellular conditions) as compared to in the cell (or under in vitro conditions selected to mimic intracellular conditions).

Exemplary cleavable linking groups include, but are not limited to, redox cleavable linking groups (e.g., —S—S— and —C(R)₂—S—S—, wherein R is H or C₁-C₆ alkyl and at least one R is C₁-C₆ alkyl such as CH₃ or CH₂CH₃); phosphate-based cleavable linking groups (e.g., —O—P(O)(OR)—O—, —O—P(S)(OR)—O—, —O—P(S)(SR)—O—, —S—P(O)(OR)—O—, —O—P(O)(OR)—S—, —S—P(O)(OR)—S—, —O—P(S)(ORk)-S—, —S—P(S)(OR)—O—, —O—P(O)(R)—O—, —O—P(S)(R)—O—, —S—P(O)(R)—O—, —S—P(S)(R)—O—, —S—P(O)(R)—S—, —O—P(S)(R)—S—, —O—P(O)(OH)—O—, —O—P(S)(OH)—O—, —O—P(S)(SH)—O—, —S—P(O)(OH)—O—, —O—P(O)(OH)—S—, —S—P(O)(OH)—S—, —O—P(S)(OH)—S—, —S—P(S)(OH)—O—, —O—P(O)(H)—O—, —O—P(S)(H)—O—, —S—P(O)(H)—O—, —S—P(S)(H)—O—, —S—P(O)(H)—S—, and —O—P(S)(H)—S—, wherein R is optionally substituted linear or branched C₁-C₁₀ alkyl); acid cleavable linking groups (e.g., hydrazones, esters, and esters of amino acids, —C═NN— and —OC(O)—); ester-based cleavable linking groups (e.g., —C(O)O—); peptide-based cleavable linking groups, (e.g., linking groups that are cleaved by enzymes such as peptidases and proteases in cells, e.g., —NHCHR^{A}C(O)NHCHR^{B}C(O)—, where R^{A} and R^{B} are the R groups of the two adjacent amino acids). A peptide based cleavable linking group comprises two or more amino acids. In some embodiments, the peptide-based cleavage linkage comprises the amino acid sequence that is the substrate for a peptidase or a protease found in cells.

In some embodiments, an acid cleavable linking group is cleavable in an acidic environment with a pH of about 6.5 or lower (e.g., about 6.0, 5.5, 5.0, or lower), or by agents such as enzymes that can act as a general acid.

In some embodiments, the linker comprises an acid labile group, e.g., hydrazone or carbamate. In some embodiments, the linker comprises an enzyme labile group e.g., maleimidecaproyl-valyl-citrullinyl-p-aminobenzylcarbamate.

The cleavable linking group can be located anywhere in the linker. For example, the cleavable linking group can be located at a terminus of the linker. In some embodiments, the cleavable linking group is located at the linker terminus distal to the affinity ligand. In some embodiments, the cleavable linking group is located at the linker terminus distal to the molecule of interest, e.g., therapeutic agent. In some embodiments, the cleavable linking group is in the linker itself. In some embodiments, the cleavable linking group connects the linker to the molecule of interest, e.g., therapeutic agent. In some embodiments, the cleavable linking group connects the linker to the affinity ligand. Thus, in some embodiments of the invention, the linker can be linked to the affinity ligand and/or the molecule of interest via a cleavable linking group.

In some embodiments, the linker can be linked to the targeting molecule and/or the molecule of interest via a non-cleavable group such as, for example, a bond, ether, thioether, and/or hydrocarbon.

In some embodiments, the linker comprises a thio-ether linkage.

In some embodiments, the linker comprises a peptide, e.g., a dipeptide, a tripeptide, a tetrapeptide, or a pentapeptide. The peptide can be optionally substituted.

In some embodiments, the linker comprises a disulfide linkage.

In some embodiments the linker comprises a self-immolative disulfide linkage.

In some embodiments, the linker is a bond.

In some embodiments, the linker is a hydrocarbon, PEG, an amino acid, a peptide, or a combination thereof. The hydrocarbon or PEG can be substituted or unsubstituted.

In some embodiments, the linker is a PEG of a molecular weight of about 200 Da to about 50 kDa.

In some embodiments, the linker comprises an optionally modified PEG and at least one amino acid, (e.g., one, two, three, four, five, six, seven, eight, nine, ten or more amino acids).

In some embodiments, the linker comprises an optionally modified PEG and two amino acids, e.g., a dipeptide.

In some embodiments, the linker comprises an optionally modified PEG and three amino acids, e.g., a tripeptide.

In some embodiments, the linker comprises a peptide of amino acid sequence Lys-Val-Gly-Ala.

In some embodiments, the linker comprises β-cyclodextran-PEG conjugate.

In some embodiments, the spacer could be a peptide such as lysine-valine-glycine-alanine.

In some embodiments, the spacer could be a β-cyclodextrin attached to a polyethylene glycol unit.

In some embodiments, the spacer could be a polyethylene glycol unit attached to a glycine or alanine which is further linked to a camptothecin cytotoxin through an ester bond.

In some embodiments, the spacer could be a combination of amino acids and polyethylene glycol unit.

In some embodiments, the spacer could be composed of amino acids and peptide bonds which also serve as cleavable linkers.

In certain embodiments, the spacer could be a hydrocarbon chain of varying length linked to the drug via a cleavable ester bond.

In certain embodiments, the spacer could be a hydrocarbon chain of varying length linked to the drug via a cleavable peptide bond.

In certain embodiments, the spacer could be a hydrocarbon chain of varying length linked to the drug via a cleavable carbamate bond.

In some embodiments, the targeting molecule is directly bonded with the capsule or linker without any spacer which could be cleavable or non-cleavable.

In additional embodiments, the capsule is further coated to protect it from environmental effects to further enhance the controlled release of the nanoparticles and/or agents. The coatings may be formed from molecules such as monosialoganglioside (G_{M1}) or polymers which are soluble, hydrophilic, have highly flexible main chains, and have high biocompatibility if used on a human or animal. These polymers are known in the art, such as, but not limited to, PEG, PEG-linked phospholipids, PEG-linked lipids, PEG-linked cholesterol, hyperbranched polyglycerol, poly(vinyl pyrrolidone), poly(acryl amide), poly(2-methyl-2-oxazoline), poly(2-ethyl-2-oxazoline), poly [N-(2-hydroxypropyl)methacrylamide], amphiphilic poly-N-vinylpyrrolidones, L-amino-acid-based biodegradable polymer-lipid conjugates, and/or polyvinyl alcohol.

The coatings my also protect the capsule during storage and/or transportation depending on the radiation wavelength, such as UV, that may cause the metallic oxide nanoparticle to start producing ROS.

Some coating components may replace a portion or all of the corresponding components in the capsule, i.e. PEG-linked lipids for the capsule lipid or PEG-linked cholesterols for the capsule cholesterols.

In some embodiments the targeting molecule is bound to a linker bound to a coating molecule. For example, a targeting molecule may be bound to an aliphatic linker which is bound to the PEG portion of a PEG-linked cholesterol, where the cholesterol portion is integrated into the hydrophobic region of the capsule.

In other embodiments, cholesterols may be added to the lipid bilayer to further increase their stability.

In yet additional embodiments, hydrophobic agents and/or metallic oxide nanomaterial may be included within the capsule.

The size of the capsule may be either nano- or microsized depending on the desired usage. If the body is animal or human, the capsule is preferably nanosized, for example from about 2 nm to about 1,000 nm, from about 5 nm to about 500 nm, or from about 10 nm to about 200 nm. If the body is a lake or river, the capsule may be either nano- or microsized, for example from about 20 nm to about 5,000 nm, from about 30 nm to about 3,000 nm, or from about 50 nm to about 2,000 nm. Conjugated capsules may be larger depending on the conjugate, such as high molecular weight PEG.

### Agents

Any agent known to those of ordinary skill in the art to be of benefit in the diagnosis, treatment, or prevention of a disease is contemplated as a therapeutic agent in the context of the present invention. Agents include pharmaceutically active compounds, hormones, growth factors, enzymes, DNA, plasmid DNA, RNA, RNAi, antisense oligonucleotides, aptamers, ribozymes, viruses, proteins, lipids, pro-inflammatory molecules, antibodies, antimicrobials, anti-inflammatory agents, anti-sense nucleotides, imaging molecules, fluorescing molecules, dyes, contrast agents, and transforming nucleic acids or combinations thereof.

Non-limiting examples of contrast agents include radiocontrast and/or magnetic resonance image (MRI) contrast agents. Radiocontrast agents may include, but not limited to, iodinated agents, barium-based agents, such as barium-sulphate, and/or gadolinium-based compounds. MRI contrast agents may include, but not limited to, paramagnetic gadolinium and/or gadolinium complexes, superparamagnetic iron and/or iron complexes such as, but not limited to, iron oxide and/or iron platinum, paramagnetic manganese and manganese complexes, and/or protein-based MRI contrast agents such as, but not limited to, β-galactosidase-activated contrast agents and/or Ca²⁺-dependent activation.

Additional agents may be any antimicrobials, antifungals, or antiparasitics known to those of ordinary skill in the art, such as, but not limited to, aminoglycosides (e.g., gentamicin, tobramycin, netilmicin, streptomycin, amikacin, neomycin), bacitracin, corbapenems (e.g., imipenem/cislastatin), cephalosporins, colistin, methenamine, monobactams (e.g., aztreonam), penicillins (e.g., penicillin G, penicillinV, methicillin, natcillin, oxacillin, cloxacillin, dicloxacillin, ampicillin, amoxicillin, carbenicillin, ticarcillin, piperacillin, mezlocillin, azlocillin), polymyxin B, quinolones, and vancomycin; and bacteriostatic agents such as chloramphenicol, clindanyan, macrolides (e.g., erythromycin, azithromycin, clarithromycin), lincomyan, nitrofurantoin, sulfonamides, tetracyclines (e.g., tetracycline, doxycycline, minocycline, demeclocyline), and trimethoprim. Also included are metronidazole, fluoroquinolones, and ritampin. Antifungals include, but are not limited to, imidazoles, triazoles, thiazoles, allylamines, polyenes, echinocandin, aurones, benzoic acid, ciclopirox olamine, 5-fluorocytosine, griseofulvin, haloprogin, tolnaftate, undecylenic acid, crystal violet, balsam of Peru, orotomides, miltefosines, and fumagillins. Miltefosine may also be used as an antiparasitic, as well as, but not limited to, nitazoxanides, melarsoprol, eflornithines, metronidazoles, tinidazoles, mebendazoles, pyrantel, thiabendazoles, diethylcarbamazines, ivermectin, niclosamides, praziquantel, albendazoles, praziquantels, rifampin, and amphotericin B. Some agents, like miltefosine, may treat more than a single class of infection.

In other embodiments, the agents may be salts, acids, bases, polymeric nanoadsorbents, dyes, or zeolites for use in water treatment.

In yet other embodiments, the agent may be a cosmetic. Cosmetic ingredients may include, but are not limited to, moisturizing agents, antioxidants, essential oils, and/or silicone containing compounds.

Non-limiting examples of moisturizing agents that can be used include, but are not limited to, amino acids, chondroitin sulfate, diglycerin, erythritol, fructose, glucose, glycerin, glycerol polymers, glycol, 1,2,6-hexanetriol, honey, hyaluronic acid, hydrogenated honey, hydrogenated starch hydrolysate, inositol, lactitol, maltitol, maltose, mannitol, natural moisturizing factor, PEG-15 butanediol, polyglyceryl sorbitol, salts of pyrollindone carboxylic acid, potassium PCA, propylene glycol, sodium glucuronate, sodium PCA, sorbitol, sucrose, trehalose, urea, and xylitol.

Other examples include acetylated lanolin, acetylated lanolin alcohol, alanine, algae extract, Aloe barbadensis, Aloe barbadensis extract, Aloe barbadensis gel, Althea officinalis extract, apricot (Prunus armeniaca) kernel oil, arginine, arginine aspartate, Arnica montana extract, aspartic acid, avocado (Persea gratissima) oil, barrier sphingolipids, butyl alcohol, beeswax, behenyl alcohol, beta-sitosterol, birch (Betula alba) bark extract, borage (Borago officinalis) extract, butcherbroom (Ruscus aculeatus) extract, butylene glycol, Calendula officinalis extract, Calendula officinalis oil, candelilla (Euphorbia cerifera) wax, canola oil, caprylic/capric triglyceride, cardamon (Elettaria cardamomum) oil, carnauba (Copernicia cerifera) wax, carrot (Daucus carota sativa) oil, castor (Ricinus communis) oil, ceramides, ceresin, ceteareth-5, ceteareth-12, ceteareth-20, cetearyl octanoate, ceteth-20, ceteth-24, cetyl acetate, cetyl octanoate, cetyl palmitate, chamomile (Anthemis nobilis) oil, cholesterol, cholesterol esters, cholesteryl hydroxystearate, citric acid, clary (Salvia sclarea) oil, cocoa (Theobroma cacao) butter, cococaprylate/caprate, coconut (Cocos nucifera) oil, collagen, collagen amino acids, corn (Zea mays) oil, fatty acids, decyl oleate, dimethicone copolyol, dimethiconol, dioctyl adipate, dioctyl succinate, dipentaerythrityl hexacaprylate/hexacaprate, DNA, erythritol, ethoxydiglycol, ethyl linoleate, Eucalyptus globulus oil, evening primrose (Oenothera biennis) oil, fatty acids, Geranium maculatum oil, glucosamine, glucose glutamate, glutamic acid, glycereth-26, glycerin, glycerol, glyceryl distearate, glyceryl hydroxystearate, glyceryl laurate, glyceryl linoleate, glyceryl myristate, glyceryl oleate, glyceryl stearate, glyceryl stearate SE, glycine, glycol stearate, glycol stearate SE, glycosaminoglycans, grape (Vitis vinifera) seed oil, hazel (Corylus americana) nut oil, hazel (Corylus avellana) nut oil, hexylene glycol, hyaluronic acid, hybrid safflower (Carthamus tinctorius) oil, hydrogenated castor oil, hydrogenated cocoglycerides, hydrogenated coconut oil, hydrogenated lanolin, hydrogenated lecithin, hydrogenated palm glyceride, hydrogenated palm kernel oil, hydrogenated soybean oil, hydrogenated tallow glyceride, hydrogenated vegetable oil, hydrolyzed collagen, hydrolyzed elastin, hydrolyzed glycosaminoglycans, hydrolyzed keratin, hydrolyzed soy protein, hydroxylated lanolin, hydroxyproline, isocetyl stearate, isocetyl stearoyl stearate, isodecyl oleate, isopropyl isostearate, isopropyl lanolate, isopropyl myristate, isopropyl palmitate, isopropyl stearate, isostearamide DEA, isostearic acid, isostearyl lactate, isostearyl neopentanoate, jasmine (Jasminum officinale) oil, jojoba (Buxus chinensis) oil, kelp, kukui (Aleurites moluccana) nut oil, lactamide MEA, laneth-16, laneth-10 acetate, lanolin, lanolin acid, lanolin alcohol, lanolin oil, lanolin wax, lavender (Lavandula angus-tifolia) oil, lecithin, lemon (Citrus medica limonum) oil, linoleic acid, linolenic acid, Macadamia ternifolia nut oil, maltitol, Matricaria (Chamomilla recutita) oil, methyl glu-cose sesquistearate, methylsilanol PCA, mineral oil, mink oil, Mortierella oil, myristyl lactate, myristyl myristate, myristyl propionate, neopentyl glycol dicaprylate/dicaprate, octyldodecanol, octyldodecyl myristate, octyldodecyl stearoyl stearate, octyl hydroxystearate, octyl palmitate, octyl salicylate, octyl stearate, oleic acid, olive (Olea europaea) oil, orange (Citrus aurantium dulcis) oil, palm (Elaeis guineensis) oil, palmitic acid, pantethine, panthenol, pan-thenyl ethyl ether, paraffin, PCA, peach (Prunus persica) kernel oil, peanut (Arachis hypogaea) oil, PEG-8 C₁₂-C₁₈ ester, PEG-15 cocamine, PEG-150 distearate, PEG-60 glyceryl isostearate, PEG-5 glyceryl stearate, PEG-30 glyceryl stearate, PEG-7 hydrogenated castor oil, PEG-40 hydrogenated castor oil, PEG-60 hydrogenated castor oil, PEG-20 methyl glucose sesquistearate, PEG40 sorbitan peroleate, PEG-5 soy sterol, PEG-10 soy sterol, PEG-2 stearate, PEG-8 stearate, PEG-20 stearate, PEG-32 stearate, PEG40 stearate, PEG-50 stearate, PEG-100 stearate, PEG-150 stearate, pentadecalactone, peppermint (Mentha piperita) oil, petrolatum, phospholipids, polyamino sugar condensate, polyglyceryl-3 diisostearate, polyquaternium-24, polysorbate 20, polysorbate 40, polysorbate 60, polysorbate 80, polysorbate 85, potassium myristate, potassium palmitate, propylene glycol, propylene glycol dicaprylate/dicaprate, propylene glycol dioctanoate, propylene glycol dipelargonate, propylene glycol laurate, propylene glycol stearate, propylene glycol stearate SE, PVP, pyridoxine dipalmitate, retinal, retinyl palmitate, rice (Oryza sativa) bran oil, RNA, rosemary (Rosmarinus officinalis) oil, rose oil, safflower (Carthamus tinctorius) oil, sage (Salvia officinalis) oil, sandalwood (santalum album) oil, serine, serum protein, sesame (Sesamum indicum) oil, shea butter (Butyrospermum parkii), silk powder, sodium chondroitin sulfate, sodium hyaluronate, sodium lactate, sodium palmitate, sodium PCA, sodium polyglutamate, soluble collagen, sorbitan laurate, sorbitan oleate, sorbitan palmitate, sorbitan sesquioleate, sorbitan stearate, sorbitol, soybean (Glycine soja) oil, sphingolipids, squalane, squalene, stearamide MEA-stearate, stearic acid, stearoxy dimethicone, stearoxytrimethylsilane, stearyl alcohol, stearyl glycyrrhetinate, stearyl heptanoate, stearyl stearate, sunflower (Helianthus annuus) seed oil, sweet almond (Prunus amygdalus dulcis) oil, synthetic beeswax, tocopherol, tocopheryl acetate, tocopheryl linoleate, tribehenin, tridecyl neopentanoate, tridecyl stear-ate, triethanolamine, tristearin, urea, vegetable oil, water, waxes, wheat (Triticum vulgare) germ oil, and ylang ylang (Cananga odorata) oil.

Non-limiting examples of antioxidants that can be used with the compositions of the present invention include acetyl cysteine, ascorbic acid polypeptide, ascorbyl dipalmitate, ascorbyl methylsilanol pectinate, ascorbyl palmitate, ascorbyl stearate, BHA, BHT, t-butyl hydroquinone, cysteine, cysteine HCl, diamylhydroquinone, di-t-butylhydroquinone, dicetyl thiodipropionate, dioleyl tocopheryl methylsilanol, disodium ascorbyl sulfate, distearyl thiodipropionate, ditridecyl thiodipropionate, dodecyl gallate, erythorbic acid, esters of ascorbic acid, ethyl ferulate, ferulic acid, gallic acid esters, hydroquinone, isooctyl thioglycolate, kojic acid, magnesium ascorbate, magnesium ascorbyl phosphate, methylsilanol ascorbate, natural botanical anti-oxidants such as green tea or grape seed extracts, nordihydroguaiaretic acid, octyl gallate, phenylthioglycolic acid, potassium ascorbyl tocopheryl phosphate, potassium sulfite, propyl gallate, quinones, rosmarinic acid, sodium ascorbate, sodium bisulfite, sodium erythorbate, sodium metabisulfite, sodium sulfite, superoxide dismutase, sodium thioglycolate, sorbityl furfural, thiodiglycol, thiodiglycolamide, thiodiglycolic acid, thioglycolic acid, thiolactic acid, thiosalicylic acid, tocophereth-5, tocophereth-10, tocophereth-12, tocophereth-18, tocophereth-50, tocopherol, tocophersolan, tocopheryl acetate, tocopheryl linoleate, tocopheryl nicotinate, tocopheryl succinate, and tris(nonylphenyl)phosphite.

In non-limiting examples, silicone containing compounds include any member of a family of polymeric products whose molecular backbone is made up of alternating silicon and oxygen atoms with side groups attached to the silicon atoms. By varying the —Si—O— chain lengths, side groups, and crosslinking, silicones can be synthesized into a wide variety of materials. They can vary in consistency from liquid to gel to solids.

The silicone containing compounds that can be used in the context of the present invention include those described in this specification or those known to a person of ordinary skill in the art. Non-limiting examples include silicone oils (e.g., volatile and non-volatile oils), gels, and solids. In certain aspects, the silicon containing compounds includes a silicone oils such as a polyorganosiloxane. Non-limiting examples of polyorganosiloxanes include dimethi-cone, cyclomethicone, polysilicone-11, phenyl trimethicone, trimethylsilylamodimethicone, stearoxytrimethylsilane, or mixtures of these and other organosiloxane materials in any given ratio to achieve the desired consistency and application characteristics depending upon the intended application (e.g., to a particular area such as the skin, hair, or eyes). A “volatile silicone oil” includes a silicone oil have a low heat of vaporization, i.e. normally less than about 50 cal per gram of silicone oil. Non-limiting examples of volatile silicone oils include: cyclomethicones such as Dow Corning 344 Fluid, Dow Corning 345 Fluid, Dow Corning 244 Fluid, and Dow Corning 245 Fluid, Volatile Silicon 7207 (Union Carbide Corp., Danbury, Conn.); low viscosity dimethicones, i.e. dimethicones having a viscosity of about 50 est or less (e.g., dimethicones such as Dow Corning 200-0.5 est Fluid). The Dow Corning Fluids are available from Dow Corning Corporation, Midland, Mich. Cyclom-ethicone and dimethicone are described in the Third Edition of the CTFA Cosmetic Ingredient Dictionary (incorporated by reference) as cyclic dimethyl polysiloxane compounds and a mixture of fully methylated linear siloxane polymers end-blocked with trimethylsiloxy units, respectively. Other non-limiting volatile silicone oils that can be used in the context of the present invention include those available from General Electric Co., Silicone Products Div., Waterford, N.Y. and SWS Silicones Div. of Stauffer Chemical Co., Adrian, Mich.

Essential oils include oils derived from herbs, flowers, trees, and other plants. Such oils are typically present as tiny droplets between the plant's cells and can be extracted by several methods known to those of skill in the art (e.g., steam distilled, enfleurage (i.e., extraction by using fat), maceration, solvent extraction, or mechanical pressing). When these types of oils are exposed to air they tend to evaporate (i.e., a volatile oil). As a result, many essential oils are colorless, but with age they can oxidize and become darker. Essential oils are insoluble in water and are soluble in alcohol, ether, fixed oils (vegetal), and other organic solvents. Typical physical characteristics found in essential oils include boiling points that vary from about 160° to 240° C. and densities ranging from about 0.759 to about 1.096.

Essential oils typically are named by the plant from which the oil is found. For example, rose oil or peppermint oil are derived from rose or peppermint plants, respectively. Non-limiting examples of essential oils that can be used in the context of the present invention include sesame oil, macadamia nut oil, tea tree oil, evening primrose oil, Spanish sage oil, Spanish rosemary oil, coriander oil, thyme oil, pimento berries oil, rose oil, anise oil, balsam oil, bergamot oil, rosewood oil, cedar oil, chamomile oil, sage oil, clary sage oil, clove oil, cypress oil, eucalyptus oil, fennel oil, sea fennel oil, frankincense oil, geranium oil, ginger oil, grape-fruit oil, jasmine oil, juniper oil, lavender oil, lemon oil, lemongrass oil, lime oil, mandarin oil, marjoram oil, myrrh oil, neroli oil, orange oil, patchouli oil, pepper oil, black pepper oil, petitgrain oil, pine oil, rose otto oil, rosemary oil, sandalwood oil, spearmint oil, spikenard oil, vetiver oil, wintergreen oil, or ylang ylang. Other essential oils known to those of skill in the art are also contemplated as being useful within the context of the present invention.

In further embodiments, the agent may be a photosensitizer. Non-limiting examples of photosensitizers may include, porphyrins and their derivatives, such as haematoporphyrin, verteporfin, and tetrakis(o-aminophenyl)porphyrin, fullerenes, porifmer sodium, phthalocyanine, phthalocyanine derivative, chlorophylls, dyes, aminolevulinic acid (ALA), silicon phthalocyanine Pc 4, m-tetrahydroxypehnylchlorine (mTHPC), mono-L-aspartyl chlorine (Npe6), benzoporphyrin, methy aminolevulinate, and ALLUMERA™.

Any of the agents can be combined to the extent such combination of agents is compatible. The agent, or combination of agents, is selected according to the objective and action desired.

Encapsulation of Nanoparticle and/or Agents

Loading of the nanomaterials and/or agents into the capsule may be passive or active. Passive loading, when the nanoparticles and/or agents are loaded during the formation of the capsule, techniques include, but are not limited to, mechanical dispersion methods, solvent dispersions methods, and/or detergent removal methods. Mechanical dispersion methods include, but are not limited to, sonication, French pressure cell: extrusion, free-thaw, lipid film hydration by shaking or freeze drying, micro-emulsification, membrane extrusion, and/or dried reconstituted vesicles. Solvent dispersions methods include, but are not limited to, ether injection/solvent vaporization, ethanol injection, and/or reverse phase evaporation methods. Detergent removal methods include, but are not limited to, dialysis, detergent removal of mixed micelles, and/or gel-permeation chromatography.

Active loading, when the agents are loaded after the formation of the capsules, techniques include, but are not limited to, direct combination of the capsule with hydrophobic agents and/or employing a pH gradient to aid in trapping of the nanoparticle and/or agents.

Ratios of lipids and/or polymers to nanomaterials and/or agents may vary by embodiment. The ratio of lipids and/or polymers to nanomaterial and/or agents will depend on the desired size of the nanoparticles and purpose of the encapsulated nanomaterial and/or agent. As the size of nanoparticle increases, the surface area made up of lipids may greatly increase in comparison to the other compounds of the composition.

Nanoparticle to lipid, and/or polymer, w/w ratio may be fined tuned to control the release of nanoparticles or agents. As the lipid to nanoparticle w/w ratio decreases, some encapsulated nanoparticles may be leaky, allowing the agents and nanoparticles to escape prior to being exposed to radiation. However, triggered release of the interior compounds will also be increased at lower lipid to nanoparticle ratios. Conversely, higher lipid to nanoparticle ratios may result in no leakage, but also slower release of the internal compounds. To determine leakage, a reporter may be encapsulated with a nanoparticle within a capsule. The reporter may be any reporter, such as horseradish peroxidase, luciferase, or a fluorescent reporter. The encapsulated reporter and nanoparticle may then be assayed, using routine methods known in the art, in the absence of the appropriate radiation to induce ROS production with the resulting read being caused by the leakage of the reporter through the capsule. Generally, as the w/w ratio of lipids to nanoparticles drops below 1:1, more leakage is seen while above 1:1 almost no leakage occurs.

The interior pH may be altered to control the rate of release of the interior compounds. For some nanoparticles an interior pH>7 may increase ROS production, leading to quicker degradation of the capsule, and quicker release of agents into the local environment. Any acid, base, or buffer may be used to adjust pH. A strong acid or base, preferably HCl or NaOH, may be used to minimize the amount needed to achieve the desired interior pH. This increase in ROS production comes from more the presence of additional hydroxides and it is believed that the valence band holes left from the excited electron convert the locally adsorbed hydroxides to hydroxyl radicals. The interior pH of the encapsulated nanoparticles may be physiological, having a pH from about 6.8 to about 7.8; may be acidic, having a pH from about 1 to about 6.8, from about 3 to about 6, from about 4 to about 6, or less than about 7; or may be basic, having a pH from about 7.8 to about 14, from about 8 to 13, from about 9 to 12, or greater than about 7.8.

As either the w/w ratio of lipid to nanoparticles and the interior pH may control the rate of release of the interior compounds or either allow or prevent leakage, both may also be altered to fine tune the encapsulated nanoparticles and agents. By way of example, to have no leakage, but still have rapid controlled release, a high lipid:NP w/w ratio along with a basic interior may be desirable. However, if a prolonged release is desirable with a bolos like effect at a given later time after administration, a low lipid:NP w/w ratio may be used with a basic interior so that the embodiment will leak the interior contents until exposed to the desired radiation, releasing the remaining contents.

### Uses

The methods and compositions of the various embodiments can be used to deliver any medicament, compositions, or particle to a desired site. The following contains a nonlimiting list of potential uses.

### Cancer Treatment

Yet another embodiment is directed to a method of treating cancer or metastasis. The method includes administering to a subject in need thereof an effective amount of the composition where the agent may be an anti-cancer treatment described herein.

For administration to a subject, the compositions described herein can be provided in pharmaceutically acceptable compositions. These pharmaceutically acceptable compositions comprise a therapeutically effective amount of one or more of the compositions described herein, formulated together with one or more pharmaceutically acceptable carriers (additives) and/or diluents. As described in detail below, the pharmaceutical compositions of the present invention can be specially formulated for administration in solid or liquid form, including those adapted for the following: (1) oral administration, for example, drenches (aqueous or non-aqueous solutions or suspensions), lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; or (9) nasally. Additionally, compounds can be implanted into a patient or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960. Both incorporated herein in their entirety.

Determination of a therapeutically effective amount is well within the capability of those skilled in the art. Generally, a therapeutically effective amount can vary with the subject's history, age, condition, sex, as well as the severity and type of the medical condition in the subject, and administration of other pharmaceutically active agents.

Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In preferred embodiments, the compositions are administered by intravenous infusion or injection.

The conjugates of the invention are also useful in combination with known anti-cancer treatments, including additional radiation or chemotherapy. The methods of the invention are especially useful in combination with anti-cancer treatments that involve administering a second drug that acts in a different phase of the cell cycle.

### Diagnosis and Monitoring

Yet another embodiment is directed to a method of the diagnosis or monitoring of various diseases, disorders or injuries. The method includes administering to a subject in need thereof an effective amount of the composition which may be any agent which is used as a contrast agent and/or the encapsulated nanoparticle may be sufficient to be used as a contrast agent itself. Determination of the amount and kind of administration of the compositions is well within the capability of those skilled in the art.

It has been demonstrated with iron oxide NPs that the lipid carriers can improve its negative contrast in MRI imaging (see Martinez-Gonzalez, R.; Estelrich, J.; Busquets, M. A., Liposomes Loaded with Hydrophobic Iron Oxide Nanoparticles: Suitable T-2 Contrast Agents for MRI. International Journal of Molecular Sciences 2016, 17 (8), 14, incorporated herein in its entirety), and may similarly improve the contrast capabilities in this system. Since encapsulation of nanoparticles, such as but not limited to ZnO, significantly reduces the toxicity of the NPs as well as encapsulated agents coupled with the concerns about side effects using traditional gadolinium-based contrast agents, this system provides an alternative that has similar or improved imaging capabilities but reduces the possible side effects associated with traditional contrast dyes.

### Antimicrobial

Yet another embodiment is directed to a method of reducing microbes, including bacteria, fungi, and/or parasites. The method includes administering to a subject in need thereof an effective amount of the composition which may have an agent which is an antimicrobe described herein.

For administration, the compositions described herein can be provided in environmentally acceptable compositions. These acceptable compositions will differ depending on the body to which the compositions are administered. In some embodiments, if administering to a subject, the compositions may be administered in a pharmaceutically acceptable composition as described above, including the determination of the therapeutically effective amount and the modes of administration. In other embodiments the compositions may be suspended in a gel or lotion and be administered locally to the skin or a wound. In further embodiments, the gel or lotion may be administered inside a subject if the wound is internal.

In other embodiments, the compositions are administered to a body of water to eliminate microbes. Administration of the compositions to the body may be upstream or up-current of a target microbe. Optionally, the compositions may be administered in or around the microbes to be eliminated. Administration of the compositions may also be more diffuse depending on the microbe being targeted. Determination of the amount and kind of administration of the compositions is well within the capability of those skilled in the art. Generally, the amount can vary with the body's size and flow, as well as the severity and type of the microbe in the body, and administration of other active agents.

### Water Treatment

In yet other embodiments, the compositions are administered to a body of water for treatment, such as the removal of contaminates, heavy metals, or to balance the pH. As described above, determination of the amount and kind of administration of the compositions is well within the capability of those skilled in the art. Generally, the amount can vary with the body's size and flow, as well as the severity and type of the contaminate, heavy metal, or imbalance in the body, and administration of other active agents. The agents may be salts, acids, bases, polymeric nanoadsorbents or zeolites

### Cosmetics

In other embodiments, the agents are cosmetics and are administered to a subject's skin in a gel, cream, or lotion. Determination of the amount and kind of administration of the compositions is well within the capability of those skilled in the art. Generally, the amount can vary with the subject's needs.

The invention is further described in detail by reference to the following experimental examples. These examples are provided for purposes of illustration only and are not intended to be limiting unless otherwise specified. Thus, the invention should in no way be construed as being limited to the following examples, but rather, should be construed to encompass any and all variations which become evident as a result of the teaching provided herein.

### EXAMPLES

### Example 1

As an exemplary nanoparticle, zinc oxide (ZnO) was selected to act as the reactive oxygen species (ROS) generator and formed into nanoparticles (NPs, together nZnO or ZnO NPs). The ZnO NPs in this study were produced by a forced hydrolysis method that has been previously reported. This particular synthesis method was chosen as it produces nZnO with a narrow visible fluorescence band that allows for fluorescent imaging using traditional fluorescent microscopy techniques, but other synthesis methods are expected to work. Briefly, zinc acetate dihydrate and polyvinylpyrrolidine (PVP) were added to diethylene glycol and brought to 80° C. Then nanopure water was added, the solution heated to 150° C., and held for 75 minutes. After cooling to room temperature, the mixture was centrifuged at 41,140×g and repeatedly washed with absolute ethanol. The pellet was dried overnight at 60° C. and pulverized with a mortar and pestle. The powder was subsequently annealed at 500° C. for 10 minutes.

NPs were characterized by a variety of techniques including: X-ray diffraction (XRD), Transmission Electron Microscopy (TEM), Fourier Transform Infrared Spectroscopy (FTIR), X-ray Photoelectron Spectroscopy (XPS), Inductively Coupled Plasma Mass Spectrometry (ICP-MS), and Energy Dispersive X-ray Spectroscopy (EDX). A Rigaku Miniflex 600 X-ray diffractometer was used for XRD measurements to verify the crystal phase and estimate the crystal size of the NPs. A JEOL JEM-2100 HR analytical transmission electron microscope was used to acquire TEM images of the nZnO to verify the crystal size and obtain the NP morphology. To evaluate the sample purity, a Physical Electronics Versaprobe XPS system and a Bruker Tensor 27 spectrometer FTIR system were utilized. For FTIR experiments, the pellet method was used where 1.5 mg of the nZnO sample was ground with 0.200 g of spectroscopic grade KBr and subsequently pressed with 8 tons of pressure for 4 minutes. For dynamic light scattering and zeta potential measurements, a Malvern Zetasizer NanoZS was utilized by suspending 0.25 mg/mL of the nZnO in nanopure water and in 130 mM NaCl. For gadolinium doped nZnO, the same synthesis procedure was used except that PVP was excluded and gadolinium acetate was added at the appropriate molar ratio with the zinc acetate dihydrate. To further evaluate the nZnO purity and to evaluate the doping concentration of gadolinium in the appropriate samples, ICP-MS was utilized. Additionally, XRD and electron diffraction patterns were collected using the same instruments to verify crystal phase purity of the gadolinium doped samples and EDX measurements were obtained to further verify the gadolinium content.

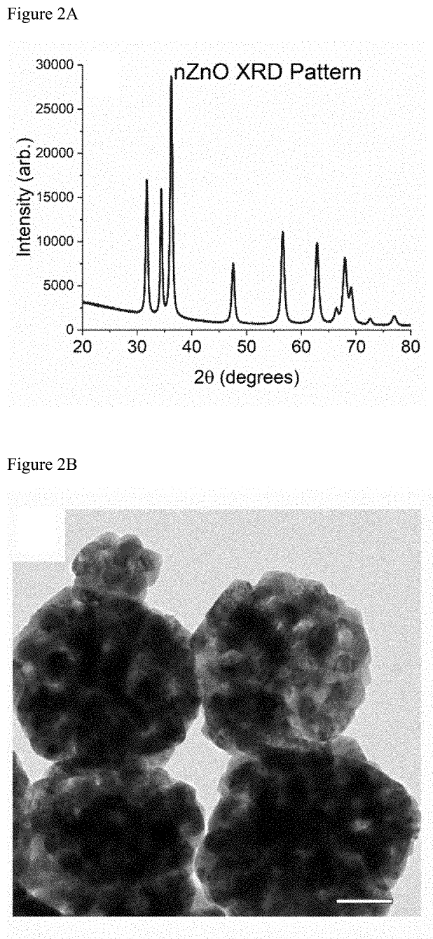

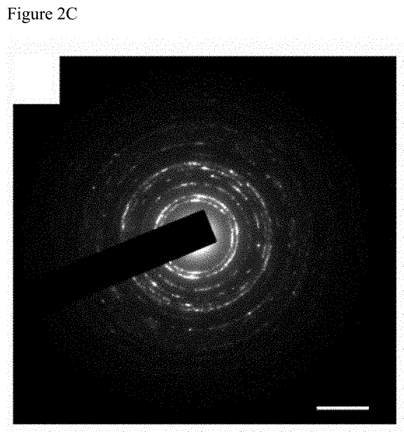

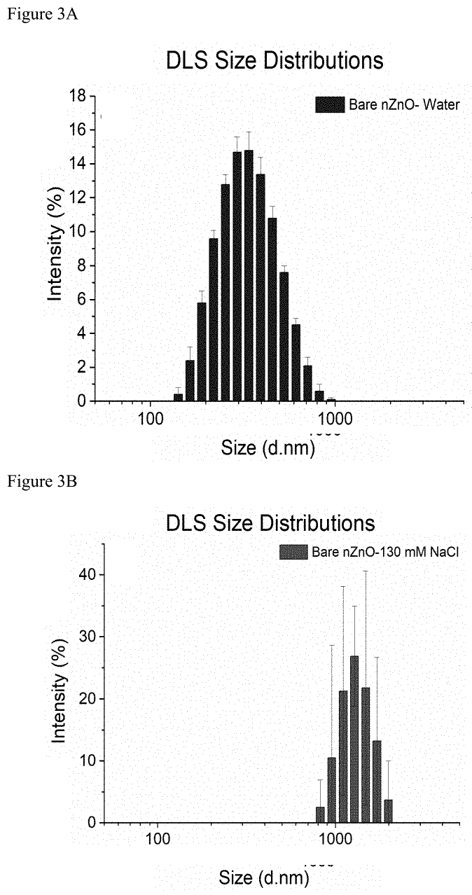

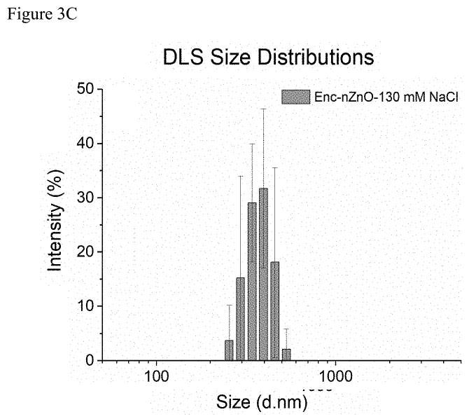

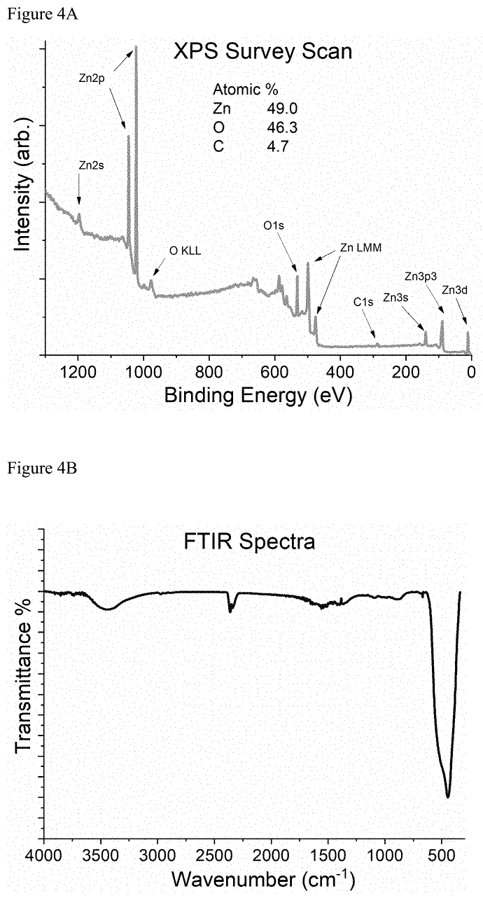

XRD measurements verified the hexagonal wurzite crystal structure, with no other phases detected and an average crystal size of 15.1±X nm (FIG. 2A). TEM images (FIG. 2B), demonstrate that the nZnO form larger aggregates of 50-400 nm comprised of smaller nZnO crystals of ˜15 nm, thereby confirming the XRD analysis. Dynamic light scattering measurements in nanopure water (FIG. 3A) and 130 mM NaCl (FIG. 3B) give the average hydrodynamic size (HDS) of the aggregated nZnO as 317±2.87/2092±149.4 (Z-avg.; d·nm) with a polydispersity index (PDI) of 0.122/0.4, respectively. XPS (FIG. 4A), FTIR (FIG. 4B), and ICP-MS measurements were used to confirm sample purity. With the exception of small amounts of carbon dioxide impurities and hydroxide formation, the nZnO are essentially pure.

### Example 2

Zinc Oxide (ZnO) nanoparticles were then encapsulated and shown to have triggered release profiles upon photo irradiation. For the initial testing of this concept, ZnO nanoparticles (nZnO) synthesized in Example 1 were utilized, however any synthesis procedure is likely to work as well as various other nanomaterials known to be photo active in photocatalytic processes. nZnO was encapsulated in a lipid bilayer comprised of phosphatidylcholine (PC) and cholesterol in a 3:1 ratio (PC to cholesterol). To start, the lipid/cholesterol mixture was suspended in chloroform with a final concentration of 13.33 mg/mL (lipid+chol) and dried under vacuum overnight to remove all the solvent. To encapsulate the NPs, nZnO was suspended in 130 mM NaCl and sonicated for 45 minutes. Simultaneously, 130 mM NaCl was added to the lipid cake (lipid+chol) for hydration and heated in a water bath to 60° C. Upon reaching 60° C., the lipids were briefly sonicated and then the nZnO solution was added for a final nZnO concentration of 4.07 mg/mL (50 mM) with various nZnO to lipid ratios. The NP/lipid/chol solution was then sonicated in a bath sonicator for 1 hour at 60° C. and stored at 4° C. until use.

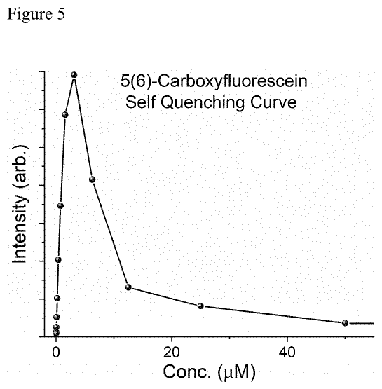

In addition to the procedures above, the same procedure was carried out to also include agents. 5(6)-carboxyfluoroscein (CF), a hydrophillic dye was added to the nZnO suspension mix at a concentration of 30 to 35 mM CF and reducing the concentration of NaCl to 100 mM, preventing osmotic pressure artifacts when resuspending the lipid coated particles in 130 mM NaCl, to simulate drug loading and to monitor the release kinetics of the vesicle upon photo irradiation. Part of the CF/NaCl solution was pH adjected to 7.4 and 9.85 by adding NaOH. At high concentrations, above about 30 mM, the fluorescent dye is self-quenched and has a very low fluorescent signal (FIG. 5). Any dye not entrapped in the vesicles was removed by repeated centrifugation, 296×g for 2 minutes, and buffer exchange, with the 130 mM NaCl buffer, until the solution was clear, and a relatively low fluorescent signal was achieved. Upon release from the vesicle into solution, the concentration of the dye is substantially decreased, allowing the dye to be highly fluorescent. The proof of encapsulation of the dye/nZnO and release was demonstrated by the addition of Triton X100, a detergent known to disrupt lipid vesicles.

Paclitaxel (PTX), a hydrophobic compound was also co-encapsulated with the nZnO nanoparticles. PTX was prepared by dissolving in dimethyl sulfoxide (DMSO) and the solution was then added to the lipid/chol while in choloroform solution. The rest of the steps were carried out as above.

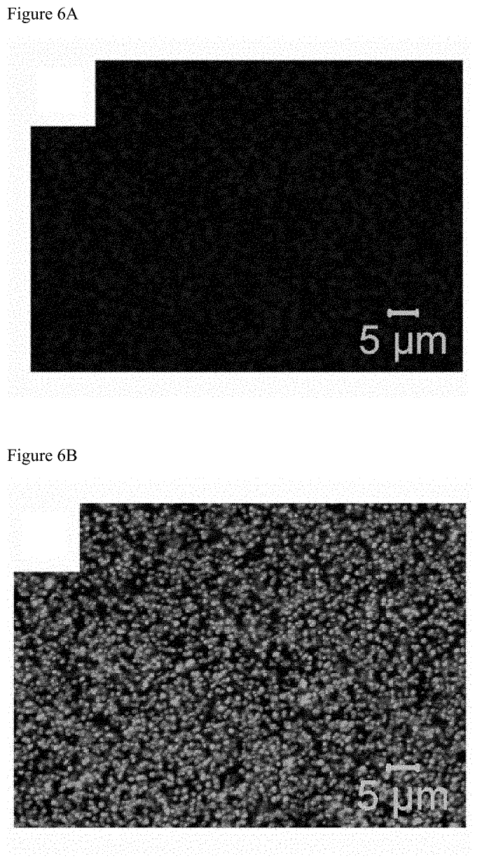

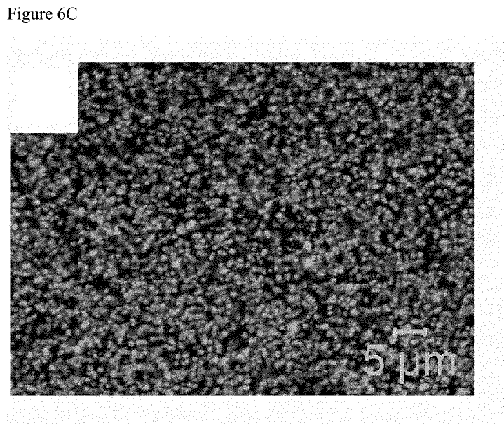

To demonstrate good encapsulation of the nZnO, confocal microscopy was employed. As seen in FIG. 6C, the fluorescent signal from the lipid capsule stain (FIG. 6B) overlays with the fluorescent nZnO (FIG. 6A), demonstrating good encapsulation of the nZnO. Dynamic light scattering measurements were subsequently performed on the encapsulated ZnO NPs (Enc-nZnO, FIG. 3B). Interestingly, a drastic decrease of the hydrodynamic size (HDS; Z-avg.; d·nm) was observed (739.5±15.5) when compared to the salt solution control and the size distribution is more similar to the free nZnO in nanopure water. This feature is attributed to the stabilization of the nZnO in the solution. This shows the hydrophilic surface of the lipid encapsulation may help prevent agglomeration of the nZnO, which is generally observed in higher ionic concentrations. This stabilization was consistent over many months, preventing the particles from agglomerating and allowing for the Enc-nZnO to be stored and used for subsequent experiments.

To determine the impact encapsulating nZnO has on toxicity, both Jurkat T cell leukemia (suspension) and T47D breast cancer (adherent) cells were treated with Enc-nZnO for 48 hours. NPs sedimentation and, more specifically, the tendency for nZnO to agglomerate and become an unstable suspension in cellular media, has been shown to result in both decreased viability of adherent cells and increased viability of suspension cell. As the Enc-nZnO have a decreased HDS and the lipid capsule prevents their agglomeration in salt solutions, their toxicity profile could conceivably be impacted and may increase the uptake of the NPs as seen in other reports. However, the lipid encapsulation could also reduce interactions of the highly reactive surface of nZnO with cells or reduce/eliminate its dissolution in cellular media which could significantly affect nZnO interactions with cells and influence their toxicity.

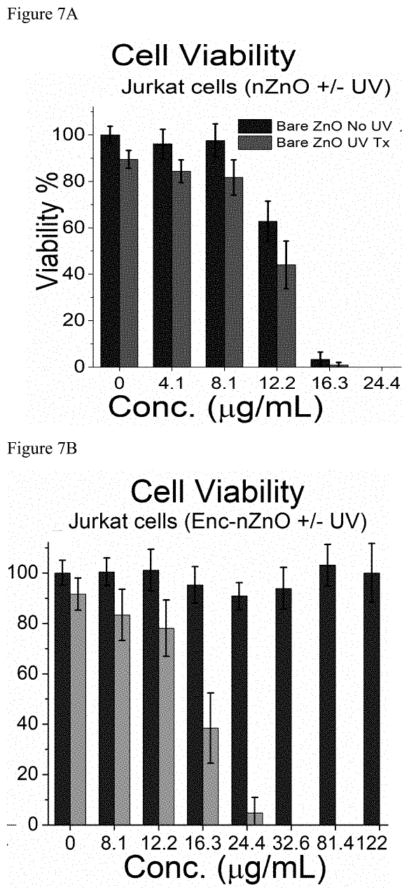

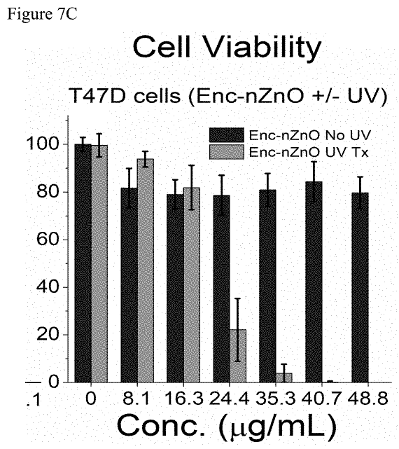

As can be seen in FIGS. 7A-C, coating nZnO with lipids effectively removes the toxicity for both Jurkat and T47D cancer cell types (No UV group: compare left bars across the 3 panels). Treatments were conducted with concentrations up to 10 times the IC₅₀ of the bare nZnO samples (˜12.2 μg/mL), and no appreciable toxicity was observed with treatment as high as 122.21 μg/mL. Further studies demonstrated the ability to trigger the release of the Enc-nZnO using phototherapy by irradiation with a 365 nm LED UV lamp.

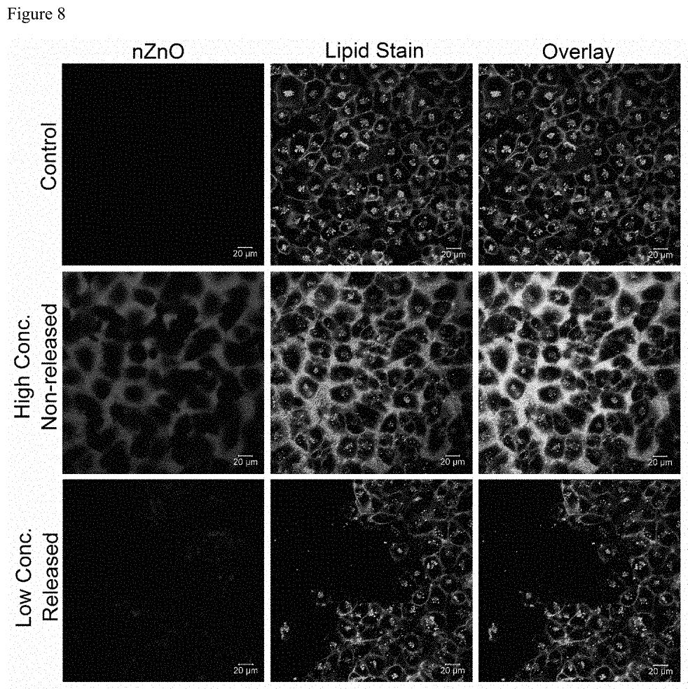

To further demonstrate both the protective nature of encapsulating nZnO and the re-establishment of the toxicity upon triggered release, confocal microscopy was utilized to image the T47D cells with Enc-nZnO after 24 hours of treatment. As can be seen in FIG. 8 (middle row), even at concentrations of Enc-nZnO that are higher than those used in the T47D viability experiments (80.1 ug/mL; 1 mM), the lipid capsule appears to protect the cells from the toxic effects of the nZnO. Even in areas with extremely high concentrations of Enc-nZnO associated with the exterior of the cells (middle row), the cell confluency is unaffected with little to no particles seen within the interior of the cell. This is in stark contrast to photo-irradiated cells treated with relatively low concentrations of the Enc-nZnO (20.3 ug/mL; 250 μM; bottom row). In many of these cells, the morphology of the membrane changes and appears to be compromised. Additionally, the membrane stain doesn't overlay with the particles as it does in FIG. 6C or in the high concentration Enc-nZnO/no UV treatment images (FIG. 8, middle row). This observation demonstrate that the particles shed the lipid capsule upon photo-irradiation. In addition, in localized areas of nZnO (bottom row), cell density is reduced and nZnO appear to have internalized into the cells. These results confirm the viability profiles and demonstrate the protective nature that the lipid capsule has on the toxicity of nZnO. Further, as it is the lipid capsule providing protection, one would also expect this same protection would also be conveyed to other toxic nanoparticles.

### Example 3

The demonstration of photo irradiation of nZnO encapsulated in a lipid bilayer and subsequent release of the CF was measured with a FluoroMax-4 spectrofluorometer with a working range of 285-750 nm. Excitation was set to 480 nm and emission spectra were collected from 490-600 nm. A self-quenching curve of CF was generated by collecting fluorescent measurements with a various concentration of free CF in 130 mM NaCl to verify that the working concentrations were within the linear fluorescent region (FIG. 5). Baseline measurements were first collected (F_{baseline}) and then a 30 W, 365 nm LED lamp was used as a UV source to cause photoexcitation of nZnO and induce the triggered release of the fluorescent dye (F). To induce full release of the encapsulated dye (Fₜₒₜₐₗ), 20 uL of 5% Triton-X stock was then added to the solution. The percentage of released CF was determined by:

% released=(F−F_{baseline})/(Fₜₒₜₐₗ−F_{baseline})×100

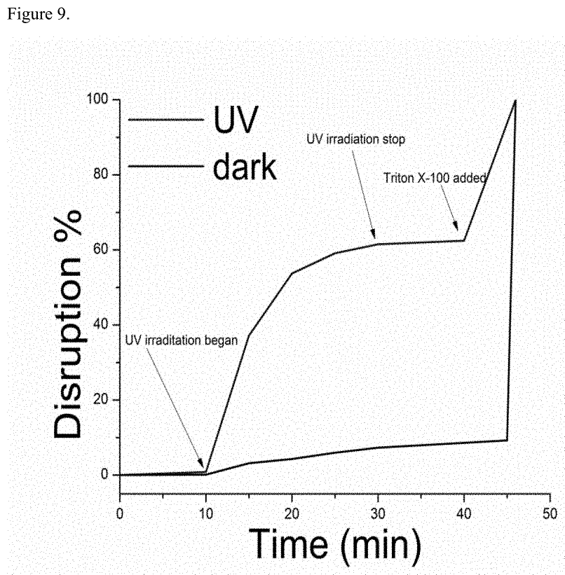

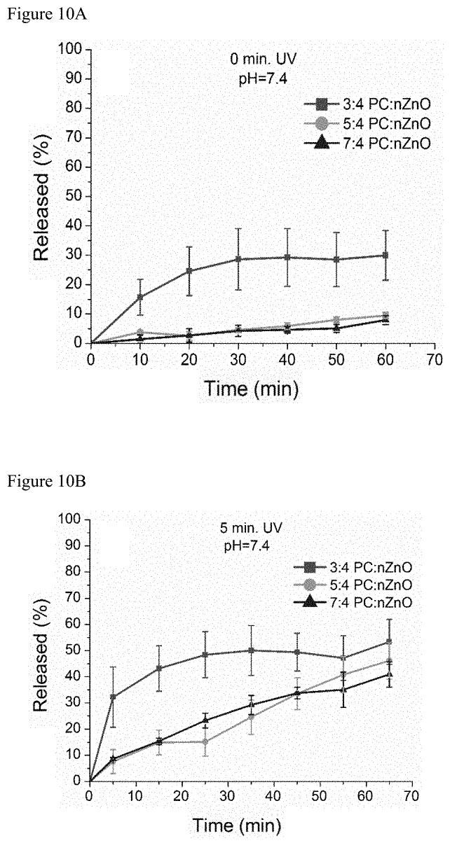

As seen in FIG. 9, the dye encapsulated in vesicles with nZnO had a substantial increase in fluorescent signal upon exposure to UV light where, in contrast, the dye encapsulated but not exposed to UV irradiation (referred to as “dark” condition) had minimal release. After the final time point of 20 minutes of irradiation, the solution sat for 10 minutes with no exposure and then Triton X was added to the solution to induce complete release. This demonstrates the maximum amount of release possible in both situations. The tests were run multiple times and demonstrate the ability to rupture the capsules and release of a hydrophilic compound upon photo irradiation. This process may also be achieved using any wavelength of light that has more energy than the band gap of ZnO (˜380 nm) such as x-rays or may be modulated to extend the working range by incorporation of different photosensitizers, allowing for the use of this delivery method of a potential chemotherapeutic in combination with phototherapy for cancer treatment.

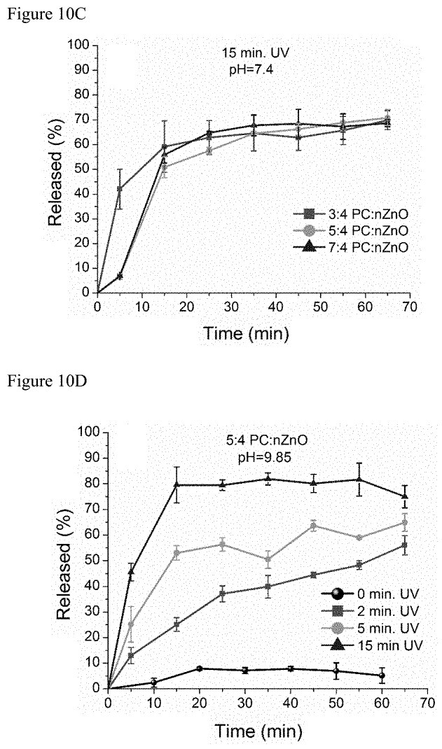

Additionally, as seen in FIG. 10A, lower lipid to nZnO ratios caused a much higher premature drug leakage even without UV exposure. However, the triggered release of the dye was drastically faster with low lipid to NP ratios (FIGS. 10B-C; 3:4 w/w) but the fluorescent signal may be partially impacted by the premature release. The higher ratios of lipid to nZnO almost entirely eliminated the premature release of the dye and reduced their release kinetics. However, after only about 1 hour post irradiation, nearly the same amount of dye was released for the 5 minute exposure (FIG. 10B), and nearly the same levels of release were obtained for the 15 minute UV exposure groups (FIG. 10C).

To try to improve upon the release kinetics, experiments were performed using a higher pH (pH=9.85) in the interior of the encapsulated nZnO/CF. The pH of the environment has been shown to impact the ROS generating capabilities of irradiated nZnO. With a high pH, more hydroxides are present, and it is believed that the valence band holes left from the excited electron convert the locally adsorbed hydroxides to hydroxyl radicals. Since the ROS generated from the irradiated nZnO is responsible for the triggered release of the lipid coating, this strategy could increase control of the overall release kinetics. FIG. 10D utilized the 5:4 PC to nZnO (w/w) ratio and the higher pH dramatically increased the release of the CF. Two minutes of UV exposure generated similar but faster release kinetics than the 5 minutes of UV exposure when using a pH of 7.4. Similarly, the release after 5 minutes of UV exposure was similar to that of the 15-minute groups using a lower pH and 15 minutes of exposure induced a faster and higher release than was achieved for any lipid to NP ratio previously.

Therefore, by adjusting both the lipid to nanoparticle ratio and the internal pH, increased control over nanoparticle and agent release may be controlled and fine-tuned for a given purpose. For example, using a high ratio and lower/neutral pH for a slow releasing, long lasting encapsulated nanoparticle may be beneficial to use in water treatment while a low ratio, high pH encapsulated nanoparticle may be beneficial to use for an imaging or therapeutic purpose.

### Example 4

To further demonstrate the ability of this mechanism, toxicity studies where performed with non-adherent Jurkat leukemia cells and adherent T47D breast cancer cells. Both cell lines were cultured in log phase using RPMI 1640 media supplemented with 10% fetal bovine serum (FBS), 1% penicillin/streptomycin, 2 mM L-glutamine, and for T47D cells, 0.2 Units/mL of bovine insulin per American Type Culture Collection (ATCC) recommendations.

To evaluate the encapsulation of nZnO within a lipid membrane, imaging experiments were performed. The Enc-nZnO sample was transferred into a Nunc Lab-Tek II Chambered Coverglass 2 that contained 130 mM NaCl. The lipids were then stained utilizing the plasma membrane stain Cell Mask Orange (Invitrogen; Carlsbad, Calif.) at a final concentration of 5 μg/mL, 30 minutes prior to imaging. For live cell imaging of T47D breast cancer cells, the cells were transferred to a Nunc Lab Tek II chamber in RPMI 1640 two days prior to imaging at a concentration of 100,000 cells/well and allowed to attach to the cover slip. After 24 hours, the media was gently aspirated and then replaced with HEPES-free, phenol-free, and phosphate-free RPMI 1640 (PPH free RPMI 1640) (custom order Thermo Fisher Scientific; Grand Island, N.Y.). The media exchange was carried out to prevent the transformation and dissolution of nZnO induced by phosphate and HEPES, respectively. Phenol red was excluded to prevent fluorescence emissions during the imaging experiments. The cells were treated with Enc-nZnO 24 hour before imaging at a final concentration of 81.4 μg/mL (1 mM) for the non-triggered released experiments. In the triggered release group, the T47D cells were treated with a final nZnO concentration of 20.3 μg/mL (250 μM). Cell Mask Orange was added to the media and cultures incubated an additional 30 minutes prior to imaging.

Confocal microscopy was used to image both the Enc-nZnO and T47D cells treated with Enc-nZnO. Images were acquired using a Zeiss 510 LSM system with the Zeiss Axiovert Observer Z1 inverted microscope and ZEN 2009 Imaging software (Carl Zeiss, Inc., Thornwood, N.Y.) utilizing different objectives and band-pass filters. Essentially pure nZnO has a low band gap of ˜3.1 eV allowing for the 405 nm laser to be used as an excitation source to produce a narrow fluorescence emission band from nZnO (410-430 nm). Using a configuration specific for the excitation and emission of the synthesized nZnO and the plasma membrane stain, images were collected with either a Plan-Apochromat 20×/NA 0.8 or Plan-Apochromat oil 63×/NA 1.4 objective. The diode (405 nm) and HeNe (543 nm) lasers were used as excitation sources, and band-pass filters of 420-480 nm and 550-647 nm were used to image the nZnO and lipid layer, respectively.

As can be seen in FIGS. 7A-C, coating nZnO with lipids effectively removes the toxicity for both Jurkat and T47D cancer cell types (No UV group: compare left bars across FIGS. 7A-C). Treatments were conducted with concentrations up to 10 times the IC₅₀ of the bare nZnO samples (˜12.2 μg/mL), and no appreciable toxicity was observed with treatment as high as 122.21 μg/mL. Further studies demonstrated the ability to trigger the release of the Enc-nZnO using phototherapy by irradiation with a 365 nm LED UV lamp. Only a few minutes of UV exposure was required to effectively restore the toxic effects of the NPs to the cancer cells (FIGS. 7A-C, left bars).

### Example 5

Since the fluorescent dye provides an excellent model for encapsulation of drugs as well as hydrophilic agents in general, Paclitaxel was chosen to model a hydrophobic agent. Paclitaxel (PTX) is commonly used in the treatment of various cancers such as non-small cell lung cancer and breast cancer. Due to its high hydrophobicity, the most widely used formulation of PTX contains polyoxyethylated castor oil, which leads to numerous side effects including hypersensitivity reactions. This drawback has led to the development of new formulations that include protein and lipid-based nanocarriers. Since lipid peroxidation is proposed as the key to the triggered release mechanism in this system, the hydrophobic lipid tails would become much more soluble in aqueous environments once peroxidation occurs. This may allow for a much faster release of PTX than in traditional lipid carriers since the drug would not be bound by the hydrophobic environment found within the lipid bilayer. In pursuit of developing a new carrier and delivery system and to demonstrate that both classes of drugs can be loaded into the compositions, both the Jurkat and T47D cell lines were treated with nZnO and Paclitaxel co-encapsulated (Enc-nZnO/PTX).

For viability assessments, both Jurkat and T47D cells were cultured following ATCC recommendations noted above. For the Jurkat T cell line, the cells were first washed with PPH free RPMI 1640 and then seeded at a concentration of 2.5×10⁵ cells/mL using the same media in a 96 well plate. For T47D cells, the cells were first seeded in the culture media at 1.0×10⁵ cells/well in a 24 well plate the day prior to treatment to allow for cell attachment. The media was then gently aspirated and replaced with the PPH free RPMI 1640 media prior to NP treatment.

The Enc-nZnO and Enc-nZnO/PTX stocks were prepared, as indicated above, at a nZnO concentration of 4.07 mg/mL (50 mM) and a physiological pH=7.2 to avoid potential effects to cellular viability using a higher pH. For the free nZnO (non-encapsulated) controls, a fresh stock was prepared for each viability experiment by first suspending the nZnO in nanopure water at a concentration of 4.07 mg/mL (50 mM) and sonicated for 20 minutes. The 2 mM PTX-DMSO solution was used to assess free PTX effects on the cells as a control. Prior to treatment, PTX-DMSO solution was added to fresh cellular media at the desired PTX concentration making the final DMSO concentration ≤0.2%. In the case of free nZnO, Enc-nZnO, and Enc-nZnO/PTX, the appropriate amount of the stock solution was added to fresh cellular media to achieve a final nZnO concentration of 1.63 mg/mL (20 mM) prior to addition.

Once treated with NPs, the cells were then cultured for 48 hours at 37° C. and 5% CO₂. For samples receiving UV irradiation, cultures were subjected to photo-irradiation 1.5 hours post NP treatment using the same 30 W, 365 nm LED lamp as in the simulated drug release studies. After optimizing the UV irradiation protocol, required UV exposure was determined to be 3 minutes for Jurkat cells and 2 minutes for T47D cells to re-establish the toxicity of the nZnO while minimizing effects to the cellular viability. Cell viability was assessed using the Alamar Blue metabolic assay. Alamar blue was added to the wells at a final concentration of 10% 44 hours post treatment and incubated for an additional 4 hours. The fluorescence intensity measurements were performed using a Biotek Synergy MX plate reader using an excitation/emission of 530/590 nm.

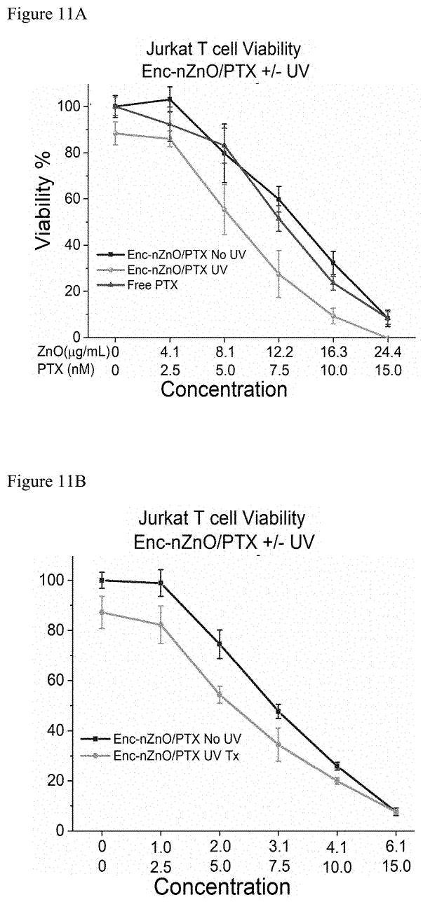

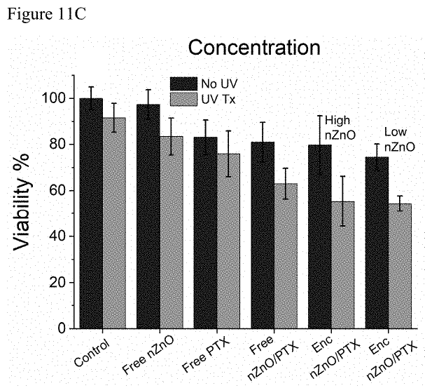

FIGS. 11A-C demonstrates that Paclitaxel and nZnO can be simultaneously delivered within the lipid carrier. In the non-triggered release viability experiments (left columns), the Jurkat T cell viability profile was similar to the free PTX treatment (FIG. 11A). While this is consistent with reports that demonstrate that lipid carriers for PTX have similar cellular viability profiles as using “free” PTX, the triggered release groups (right columns) showed a trend towards improvement in efficacy. FIG. 11A used a higher ratio of nZnO to PTX. The drop in the viability is due to a combined synergistic PTX and nZnO effect at concentrations of nZnO that were previously shown to affect the viability of cells (>12.2 μg/mL; FIGS. 7B-C). While simultaneous exposure to both nZnO and PTX could be beneficial in the treatment of cancer, the efficacy when only PTX effects are evident was assayed for comparison. To accomplish this, PTX was loaded with a lower nZnO to PTX ratio (FIG. 11B) to mitigate direct toxicity effects from nZnO. Even with concentrations of nZnO that do not impact the viability of the cells, an increase in toxicity of the triggered release group was still achieved (FIG. 11B). These findings demonstrate that the triggered release of PTX from the lipid carrier surprisingly improves the efficacy of the drug (see FIG. 11C for controls and comparison of the two ratios).

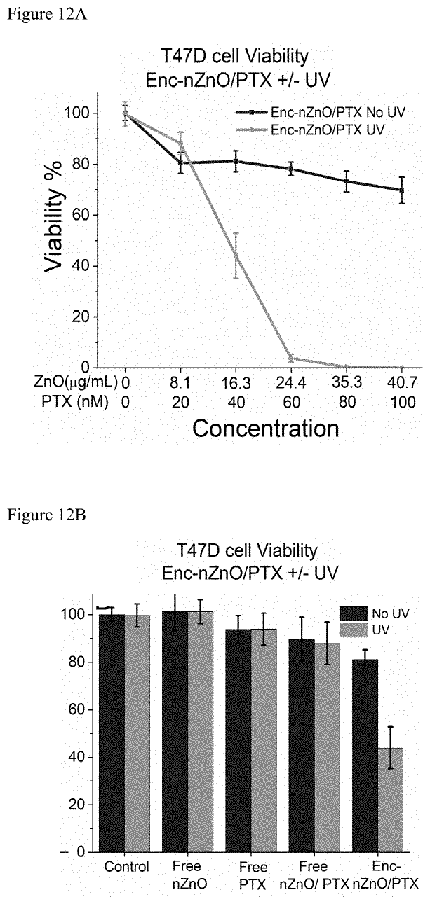

To validate these findings, experiments were repeated in another cell type. Surprisingly, T47D breast cancer cells demonstrated an even larger separation in viability than Jurkat cells (FIG. 12A). Interestingly, at or above the 40 nM PTX concentration, there was an extreme difference in the viability of the T47D cells when comparing the UV vs. No UV groups. Previous experiments with Enc-nZnO (no PTX; FIG. 57C) showed no appreciable difference in toxicity between the control and the irradiated groups at concentrations of nZnO up to 16.3 μg/mL (200 μM). However, a ˜40% difference was noted in the Enc-nZnO/PTX groups at this nZnO concentration (FIG. 12A) due to surprisingly enhanced effects from co-treatment with both nZnO and PTX. As seen with the controls in FIG. 11B, the free nZnO had no effect on the viability profile, and the treatment with free 40 nM PTX had a somewhat similar effect as the non-triggered release Enc-nZnO/PTX group. Also, co-treatment with both the free nZnO and PTX had no appreciable increase in toxicity, when compared to the PTX treatment alone. Thus, we ruled out the possibility of additive or synergistic effects from nZnO and PTX at these concentrations.

Therefore, this dramatic reduction in the viability is most likely due to the fact that the T47D cell line is adherent whereas the Jurkat cells are a suspension cell line. The dispersion stability of the NPs most likely plays a role between these two cancer cell model systems. Previous reports on nZnO and other NPs have demonstrated that overtime the particles settle to the bottom of the culture wells, affecting the dosimetry which can impact cell viability. However, this alone cannot explain the differences in toxicity because the same nZnO were used in the control groups. Since the phototherapy was conducted 1.5 hours after the nZnO treatment, many of the particles likely settled to the bottom of the well before the irradiation occurred. Indeed, the confocal images of the Enc-nZnO treated T47D cells (FIG. 8) demonstrate a high accumulation of the encapsulated NPs in the local vicinity of the cells, further strengthening this argument. When the PTX was rapidly released from the carrier due to photo-irradiation, this created a much higher local concentration of the free drug near the cells that, in turn, created a much higher cytotoxic response. These results also support the concept that the triggered release of the same overall drug concentration near cancer cells can improve the efficacy of the drug in the treatment of cancer.

This further supports the benefits of localizing encapsulated nanomaterial to their target within a body.

### Example 6

X-rays have also been used to create ROS when gadolinium doped ZnO nanoparticles were irradiated, we believe that this same procedure would be feasible upon X-ray exposure and the combination of all three (nZnO/chemo drugs/radiation therapy) would provide significant toxicity to cancer cells and could potentially be a novel alternative to combating cancer and potentially drug resistant cancer cell lines.

Gadolinium doped ZnO nanoparticles (Gn-nZnO) were made as in Example 1, with the following modification. Gadolinium acetate, at the appropriate molar ratio, was added to the zinc acetate dihydrate in place of the polyvinylpyrrolidine during ZnO nanoparticle synthesis. The nanoparticles were then encapsulated (enc-Gn-nZnO) using the same encapsulating method as in Example 1. The enc-Gn-nZnO nanoparticles were also encapsulated with CF and show release of agents when exposed to X-rays. This would provide benefits over the current art. The capsule would protect the body from the side effects of traditional gadolinium-based CT and MRI agents and increase contrast. Further, by conjugating targeting ligands to the capsule, a higher localized concentration of the enc-Gn-nZnO may be achieved, allowing for lower dosages to be administered. Additionally, drugs may be administered at the same time, potentially reducing the number of times a subject is exposed to X-rays.

What is claimed is:

1. A capsule for controlled release of loaded contents, comprising:
an outer layer surrounding the contents, wherein said capsule becomes disrupted after exposure to irradiation so that the contents are released; and
contents comprising a semiconductor nanoparticle loaded within the outer layer, wherein said nanomaterial generates reactive oxygen species (ROSs) when exposed to irradiation.

2. The outer layer of the claim 1, wherein the outer layer comprises at least one of one or more lipids and/or polymers.

3. The encapsulated nanoparticle composition of claim 1, further comprising:
at least one coating at least partially surrounding the exterior of the outer layer, wherein said coating protects said capsule from environmental effects.

4. The semiconductor nanoparticle of claim 1, wherein the semiconductor nanoparticle is a quantum dot.

5. The quantum dot of claim 4, wherein the quantum dot comprises at least one of: Si, Ge, CdTe, PbS, PbS₂, CdSe, CdS, InAs, InP, PbSe, CuInS, ZnS, CdSₓSe₁₋ₓ, and/or graphene.

6. The quantum dot of claim 4, further comprising a dopant.

7. The dopant of claim 6, wherein the dopant is one or more of boron and/or phosphorous.

8. The semiconductor nanoparticle of claim 1, wherein the semiconductor nanoparticle is a metal oxide.

9. The metal oxide of claim 8, wherein the metal comprises at least one of: zinc, gold, silver, platinum, titanium, magnesium, calcium, zirconium, iron, vanadium, nickel, copper, aluminum, strontium, barium, hafnium, and/or cerium, and/or silicon.

10. The semiconductor nanoparticle of claim 8, further comprising an association particle.

11. The association particle of claim 10, wherein the particle is at least one of: gold, silver, iron, gadolinium, lanthanum, and/or any lanthanide.

12. The capsule of claim 1, further comprising:
at least one conjugated targeting molecule conjugated to the surface of the outer layer, wherein the at least one targeting molecule will bind to a target.

13. The coating of claim 3, wherein the at least one coating is made of a lipid bilayer, monosialoganlioside, and/or a polymer, wherein said polymer has a highly flexible main chain, soluble, and is partially hydrophilic.

14. The coating of claim 13, wherein the coating is a lipid bilayer comprising an organism's membrane.

15. The contents of claim 1, further comprising an agent.

16. The outer layer of claim 1, further comprising an agent.

17. The capsule of claim 1, wherein the capsule is about 2 nm to about 5,000 nm.

18. A method of releasing the contents of a capsule, comprising:
obtaining the capsule of claim 1; and
irradiating said capsule, wherein the radiation causes the semiconductor nanoparticles to generate reactive oxygen species, and
wherein said reactive oxygen species disrupt said capsule allowing the substantial release of the contents.

19. The radiation of claim 18, wherein the radiation is electromagnetic.

20. The radiation of claim 18, wherein the radiation is one or more of ultraviolet, X-ray, and/or visible light.

21. The radiation of claim 18, wherein the radiation is ultrasound.

22. The radiation of claim 21, wherein the ultrasound is high intensity.

23. The radiation of claim 21, wherein the ultrasound is low intensity.

24. The method of claim 18, further comprising administrating a capsule for controlled release of loaded contents, comprising:
an outer layer surrounding the contents, wherein said capsule becomes disrupted after exposure to irradiation so that the contents are released; and
contents comprising a semiconductor nanoparticle loaded within the outer layer, wherein said nanomaterial generates reactive oxygen species (ROSs) when exposed to irradiation, to a body prior to irradiation.

25. The body of claim 24, wherein said body is animal.

26. The body of claim 24, wherein said body is human.

27. The body of claim 24, wherein said body is a body of water.

28. The method of claim 24, further comprising treating the body.

29. The capsule of claim 18, further comprising an agent, wherein said agent treats and adverse condition.

30. The agent of claim 29, wherein the agent is a cancer treatment.

31. The agent of claim 29, wherein the agent is a water treatment.

32. The agent of claim 29, wherein the agent is an antimicrobial.

33. The agent of claim 29, wherein the agent is a cosmetic.

34. The agent of claim 29, wherein the agent is an imaging and/or contrasting agent.

35. A method of making an encapsulated nanoparticle, comprising:
synthesizing a capsule or capsule precursor;
synthesizing a semiconductor nanomaterial;
introducing said semiconductor nanomaterial to said capsule or capsule precursor;
encapsulating said semiconductor nanomaterial within said capsule.

36. The encapsulating step of claim 35, wherein the encapsulation is active.

37. The encapsulating step of claim 35, wherein the encapsulation is passive.

38. The method of claim 35, further comprising:
encapsulating at least one agent within the capsule.

39. The method of claim 35, further comprising:
conjugating a targeting molecule to the capsule, wherein the targeting molecule is exposed to the exterior of the capsule.

40. The method of claim 39, wherein said conjugating is a covalent bond between the targeting molecule and a linker and a covalent bond between the linker and a lipid, wherein said lipid is integrated into the capsule.

41. The method of claim 35, further comprising:
coating the capsule, wherein said coating is made of a lipid bilayer, monosialoganlioside, and/or a polymer, wherein said polymer has a highly flexible main chain, soluble, and is partially hydrophilic.
